(19) United States
(12) Patent Application Publication
(10) Pub. No.: US 2018/0052161 A1
(43) Pub. Date: Feb. 22, 2018

(54) BIOMARKERS FOR MALARIA DIAGNOSIS

(71) Applicants: The United States of America, as represented by the Secretary, Dept. of Health and Human Services, Bethesda, MD (US); Emory University, Atlanta, GA (US)

(72) Inventors: Ya Ping Shi, Decatur, GA (US); Laurence Slutsker, Atlanta, GA (US); Youngja Park, Atlanta, GA (US); Dean P. Jones, Atlanta, GA (US); Loukia Lili-Williams, Atlanta, GA (US)

(73) Assignees: The United States of America, as represented by the Secretary, Dept. of Health and Human Services, Bethesda, MD (US); Emory University, Atlanta, GA (US)

(21) Appl. No.: 15/558,545

(22) PCT Filed: Mar. 16, 2016
(86) PCT No.: PCT/US2016/022666

§ 371 Date: Sep. 14, 2017

Related U.S. Application Data
(60) Provisional application No. 62/133,818, filed on Mar. 16, 2015.

(51) Int. Cl. G01N 33/569

(52) U.S. Cl. CPC G01N 33/56905; G01N 2333/445

(57) ABSTRACT

Disclosed herein are methods of detecting Plasmodium in a subject (for example, presence of Plasmodium parasite) by detecting the presence and/or amount of one or more metabolites in a sample from the subject. In some embodiments, the methods include detecting in the sample one or more metabolites listed in Table 1, Table 2, and/or Tables 5-8. The amount of the one or more metabolites in the sample is compared to the amount of the one or more metabolites in a control and presence of Plasmodium is determined if the amount of the one or more metabolites is different (for example statistically significantly increased or decreased) compared to the control.

### CROSS REFERENCE TO RELATED APPLICATION

This claims the benefit of U.S. Provisional Application No. 62/133,818, filed Mar. 16, 2015, which is incorporated herein by reference in its entirety.

### ACKNOWLEDGMENT OF GOVERNMENT SUPPORT

This invention was made with government support under grant numbers HL113451, ES009047, ES019776, and AG038746 awarded by the National Institutes of Health. The government has certain rights in the invention.

### FIELD

This disclosure relates to methods of detecting presence of malaria parasites in a subject, particularly by detecting presence and/or amount of Plasmodium metabolites or other low molecular weight molecules in a sample.

### BACKGROUND

Among four species of human malaria parasites, Plasmodium falciparum is responsible for most malaria-attributed morbidity and mortality. Over the past decade, successful scale-up of malaria control has resulted in substantial reductions in malaria cases and deaths. As malaria transmission decreases due to control efforts, the epidemiology of malaria may change; for example, an increasing proportion of infections at the community level may be asymptomatic and of low parasite density (Harris et al., Malar. J. 9:254, 2010; Mosha et al., Malar. J. 12:221, 2013). Current malaria diagnostic tools include: 1) parasite detection by microscopic examination of blood smears, 2) antigen-based rapid diagnostic tests (RDTs), and 3) sensitive DNA-based assays.

### SUMMARY

Currently available diagnostic methods require blood sampling (for example, by finger prick), and their implementation has been limited by either their labor- or time-intensive nature and/or their requirement for specialized training and skills (microscopic method), moderate sensitivity (RDTs, microscopy), or high cost of sample preparation and supporting infrastructure needed (DNA-based methods). For programs aiming to reduce transmission by further decreasing the parasite reservoir in humans through large scale screening approaches to detect and then radically cure asymptomatic low density malaria infections, a sensitive, low-cost, simple, and field-deployable non-invasive diagnostic tool would be very useful at the community level; however, currently available tools cannot meet this challenge.

As disclosed herein the inventors have identified molecules (for example low molecular-weight metabolites), such as Plasmodium-specific waste products, in the supernatant from an erythrocyte culture system and/or from saliva or urine samples from individuals infected with Plasmodium. Metabolites and other low molecular weight molecules (such as short peptides) are candidates for potential biomarkers for development of non-invasive and sensitive malaria diagnostic tools because they can be secreted into urine, saliva, or sweat in malaria infected subjects. As disclosed herein, high-resolution metabolomics (HRM) produced a relatively comprehensive and quantitative analysis of Plasmodium-specific metabolites in supernatant from a parasite-infected culture system and from saliva and urine samples from Plasmodium-infected individuals. Thus, disclosed herein are methods for detection of Plasmodium or diagnosis of Plasmodium infection that include detecting presence and/or amount of one or more of the disclosed metabolites in a sample from a subject.

Disclosed herein are methods of detecting presence of Plasmodium in a sample (for example, Plasmodium infection) by detecting the presence (such as Plasmodium-specific metabolites) in a sample from the subject. In some embodiments, the methods include detecting in the sample one or more (such as 1, 2, 3, 4, 5, 10, 20, or more) of the metabolites listed in Table 1, Table 2, Table 5, Table 6, Table 7, and/or Table 8. The amount of the one or more metabolites in the sample is compared to the amount of the one or more metabolites in a control and presence of Plasmodium is determined if the amount of the one or more metabolites is different (for example, a statistically significant increase or decrease) compared to the control.

The foregoing and other features of the disclosure will become more apparent from the following detailed description, which proceeds with reference to the accompanying figures.

### BRIEF DESCRIPTION OF THE DRAWINGS

FIGS. 1A and 1B are diagrams showing mapping of 439 matched features covering KEGG human (FIG. 1A) and Plasmodium (FIG. 1B) metabolic pathways.

FIG. 2 is a Manhattan plot of 3270 features from a metabolome-wide association study of metabolites from supernatants from Plasmodium-infected and non-infected erythrocyte cell cultures. The features from duplicate runs were averaged, log₂ transformed, and quantile normalized to identify significant features using false discovery rate (FDR). The horizontal line represents FDR q=0.05. Metabolites above the line were significant (n=1025) between the two groups.

FIG. 3 is a two-way hierarchical cluster analysis (HCA) on FDR significant features of supernatants between infected and non-infected cultures. HCA was performed using the 1025 metabolites at FDR q=0.05. The analysis utilized the samples from all time points to separate two groups in top bar.

FIG. 4 is a graph showing the number of significant features (FDR q=0.05) at each time point.

FIGS. 5A and 5B are graphs showing concentration of arginine (FIG. 5A) and isoleucine (FIG. 5B) during culture. White bars are supernatant from non-infected culture; black bars are supernatant from Plasmodium-infected culture.

FIGS. 6A-6D are graphs showing concentration of 3-methylindole (FIG. 6A), succinylacetone (FIG. 6B), S-methyl-L-thiocitrulline (FIG. 6C), and O-arachidonoyl glycidol (FIG. 6D) during culture. White bars are supernatant from non-infected culture; black bars are supernatant from Plasmodium-infected culture.

FIG. 7 is a graph showing increase in phosphorylcholine concentration during 48 hours culture. White bars indicated supernatants from plasmodium non-infected culture and black bars represent supernatants from plasmodium infected culture.

FIGS. 8A and 8B are boxplots of 3-methylindole (FIG. 8A) and succinylacetone (FIG. 8B) at each time point during 48 hours culture.

FIGS. 9A-9F are spectra of 3-methylindole (FIG. 9A), 3-methylindole after addition to cell supernatant (FIG. 9B), 3-methylindole in cell supernatant (FIG. 9C), and succinylacetone (FIGS. 9D-9F). For FIGS. 9A-9C, the top panel is total ion chromatography, the middle panel is MS, and the bottom panel is MS/MS. For FIG. 9D, the top panel is total ion chromatography and the bottom panel is MS on itself. For FIG. 9E, the top panel is MS/MS on itself and the bottom panel is MS/MS after its addition to cell supernatant. FIG. 9F is MS/MS on cell supernatant without chemical addition.

FIGS. 10A and 10B are Manhattan plots showing the significant metabolites (points above the dotted line) in saliva (FIG. 10A) and urine (FIG. 10B) samples that are associated with parasitemia levels in human subjects, controlling for age and gender and using the data from Group 1 as an example. These metabolites were the ones identified by the linear regression model in Method 1 described below and they are plotted in relation to retention time.

FIGS. 11A-11D are Principal Component Analysis (PCA) 3D plots showing the separation of the healthy vs. the malaria samples in saliva (FIG. 11A) and in urine (FIG. 11B) samples for Group 1, and in saliva (FIG. 11C) and urine (FIG. 11D) samples for Group 2 top metabolites. The separation was based on the intensity levels of the top metabolites identified by combining the two independent classification methods (linear regression model and partial-least squares regression model). Taking into account the small amount of metabolites, the separation was good in both the saliva and urine in either Group 1 or Group 2.

FIGS. 12A-12D are ROC curves and associated AUC values using various number of metabolite features (m/z) from the top 10 most significant features. FIGS. 12A and 12C are ROC curves using the top metabolites in saliva for Group 1 and Group 2, respectively. FIGS. 12B and 12D are ROC curves using the top metabolites in urine for Group 1 and Group 2, respectively.

### DETAILED DESCRIPTION

Disclosed herein are Plasmodium falciparum specific low molecular weight metabolites that can be used as biomarkers for malaria diagnosis. Using supernatant samples from in vitro erythrocytic stage cultures, or saliva or urine samples from Plasmodium-infected individuals, numerous molecules were identified as markers of malaria infection, such as those listed in Tables 1, 2, and 5-8, including but not limited to 3-methylindole, succinylacetone, S-methyl-L-thiocitrulline, O-arachidonoyl glycidol, isoleucine, and arginine.

Use of HRM is an advantageous approach to identify putative biomarkers for a complex disease like malaria, since Plasmodium parasites divert nutrients toward proliferating parasite cells while the host cells try to maintain homeostasis and deal with metabolic changes during the parasites' intraerythrocytic life cycle (Lakshmanan et al., Mol. Biochem. 175:104-111, 2011; LeRoux et al., Trends Parasitol. 25:474-481, 2009). As shown herein, this approach allows for the identification of biomarkers associated with Plasmodium. Previous studies also showed that the analytic capabilities of metabolomics can measure the relative levels of all metabolites simultaneously in in vitro and in vivo systems, including in malaria parasite infection (Park et al., Toxicol. 295:47-55, 2012; Yu et al., J. Proteome Res. 12:1419-1427, 2013; Olszewski et al., Cell Host Microbe 5:191-199, 2009; Sana et al., PLoS One 8:e60840, 2013).

Using the outcome of HRM of in vitro Plasmodium culture samples, KEGG mapping was performed in this study. Significant features (n=1025) were identified in both human and Plasmodium metabolic pathways to distinguish which metabolic compounds are being utilized by both. Surprisingly, 439 metabolites were found to be used in both human and Plasmodium metabolic pathways. However, despite the similarities in this large number of metabolites, the pathways in which these metabolites are found are likely to be different. Meanwhile, of the 586 unmatched features, a number (including 3-methylindole, succinylacetone, S-methyl-L-thiocitrulline and O-arachidonoyl glycidol) were found to be potential biomarkers from the parasite during the erythrocytic stage culture system. This was based on the fact that the ion intensities increased with culture time, suggesting a positive association between relative quantity of these molecules and level of parasitemia. HRM coupled with network and pathway analysis using the significant metabolites from culture supernatants of infected erythrocytes and incorporating the broader human and malaria parasite metabolomic knowledge identified parasite-specific biomarkers.

Additionally, HRM was performed on saliva and urine samples from malaria-infected and non-infected subjects. This analysis identified 4,031 metabolite features from saliva and 3,190 metabolite features from urine. These features were subsequently refined to 20 potential biomarkers of greatest interest from saliva (Tables 5 and 6) and 18 potential biomarkers of greatest interest from urine (Tables 7 and 8).

These findings provide for development of new malaria diagnostic tools.

### I. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means include A, or B, or A and B. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

In order to facilitate review of the various embodiments, the following explanations of certain terms are provided:

Control:

A “control” refers to a sample or standard used for comparison with an experimental sample. In some embodiments, the control is a sample obtained from a healthy subject (such as a non-Plasmodium-infected subject) or from in vitro culture without pathogen inoculation. In some embodiments, the control is a historical control or standard reference value or range of values (such as a previously tested control sample, such as a group of samples from subjects who are not infected with Plasmodium). A control may also be a threshold level or cutoff value, such as amount of a biomarker that indicates a presence of a condition (such as presence of Plasmodium or malaria infection).

Detecting:

Determining presence and/or amount of a molecule (such as a metabolite) in either a qualitative or quantitative manner. Exemplary detection methods include mass spectrometry, immunoassay, and aptamer- or antibody-based assays.

Low Molecular Weight:

Molecules with a molecular weight of about 1500 Da or less. In some examples, low molecular weight molecules include metabolites or other molecules of about 500-1500 Da (such as about 200-800 Da, about 100-500 Da, about 500-1000 Da, or about 750-1500 Da). In other examples, a low molecular weight molecule is a molecule of at least about 50, 100, 150, 200, 250, 300, 350, 400, 450, 500, 550, 600, 650, 700, 750, 800, 850, 900, 950, 1000, 1100, 1200, 1300, 1400, or 1500 Da.

Malaria:

Malaria is a parasitic infection of humans and non-human primates by the Plasmodium species P. falciparum, P. vivax, P. ovale, P. malariae, and P. knowlesi. Humans become infected following the bite of an infected anopheline mosquito, the host of the malarial parasite. Malaria occasionally occurs in humans following a blood transfusion or subsequent to needle-sharing. Clinical manifestations of malarial infection may include blackwater fever, cerebral malaria, respiratory failure, hepatic necrosis, and/or occlusion of myocardial capillaries. Additional Plasmodium species infect other hosts, such as rodents (P. berghei, P. chabaudi, P. vinckei, and P. yoelii), other mammals, birds, and reptiles.

Metabolite:

A biomolecule that has a functional and/or compositional role in a biological system, and which is not a molecule of DNA, RNA, or protein. Examples of metabolites include lipids, carbohydrates, vitamins, co-factors, pigments, amino acids, nucleotides, small peptides (for example peptides of 2-5 amino acids), and so forth. Metabolites can be obtained through the diet (consumed from the environment) or generated within an organism (for example, by a catabolic or anabolic pathway). Genes and proteins exist in large part to break down, modify, and synthesize metabolites. Metabolites are not only directly responsible for health and disease, but their presence in a biological system is the result of a variety of factors including genes, the environment, direct nutrition, and/or disease state (for example, presence of a pathogen or parasite). By detecting the presence and/or amount of one or more metabolites in a biological sample, for instance using the methods described herein, presence of a pathogen (such as a malaria parasite) can be detected.

Sample:

A specimen, such as a cell, a collection of cells (e.g., cultured cells) or supernatants from cells (e.g., supernatants from cultured cells), a tissue sample, a biopsy, or an organism. Samples also include blood and blood products (e.g., whole blood, plasma, or serum) and other biological fluids (e.g., tears, sweat, saliva and related fluids, urine, tears, mucous, and so forth). Biological samples may be from individual subjects (e.g., humans, non-human primates, rodents, or veterinary subjects) and/or archival repositories. The samples may be acquired directly from the individuals, from clinicians (for instance, who have acquired the sample from the individual), or directly from archival repositories.

Subject:

Living multi-cellular vertebrate organisms, a category that includes both human and non-human mammals. Subjects include veterinary subjects, including livestock such as cows and sheep, rodents (such as mice and rats), and non-human primates.

### II. Methods of Detecting Plasmodium Infection

Disclosed herein are metabolites identified by high resolution metabolomics that are increased or decreased in cells infected with Plasmodium (or samples, such as supernatants, from cells infected with Plasmodium) as compared with non-infected cells (or samples, such as supernatants, from non-infected cells) or in samples (such as blood, plasma, serum, saliva, urine, or sweat) from subjects infected with Plasmodium as compared to samples from non-infected subjects. The metabolites are used in methods of detecting presence of Plasmodium in a sample (for example a sample from an individual infected with or suspected to be infected with Plasmodium) or in the subject from which the sample was obtained. In some embodiments, the methods include detecting one or more metabolites (such as 2, 3, 4, 5, 10, 15, 20, or more) of the metabolites in Table 1 and/or Table 2 in a sample from a subject. In other embodiments, the methods include detecting one or more metabolites (such as 2, 3, 4, 5, 6, 7, 8, 9, 10, 11, 12, 13, 14, 15, 16, 17, 18, 19, 20, or more) of the metabolites in any one of Tables 5-8,or a combination thereof. In some examples, a change in the amount (or relative amount) of one or more metabolites in a sample (such as an increase or a decrease) compared to a control indicates the presence of Plasmodium in the sample, or in the subject from which the sample was obtained.

The methods described herein may be used for any purpose for which detection of metabolites of Plasmodium, is desirable, including diagnostic and prognostic applications, such as in laboratory and clinical settings. Appropriate samples include any conventional biological samples, including clinical samples obtained from a human or veterinary subject. Suitable samples include all biological samples useful for detection of Plasmodium metabolites in subjects, including, but not limited to, cells (such as erythrocytes), tissues, autopsy samples, bone marrow aspirates, bodily fluids (for example, blood, serum, plasma, urine, sweat, saliva, cerebrospinal fluid, middle ear fluids, breast milk, bronchoalveolar lavage, tracheal aspirates, sputum, oral fluids, nasopharyngeal aspirates, oropharyngeal aspirates), oral swabs, eye swabs, cervical swabs, vaginal swabs, rectal swabs, stool, and stool suspensions. The sample can be used directly or can be processed, such as by adding solvents, preservatives, buffers, or other compounds or substances. In other examples, the sample is concentrated, for example by centrifugation (for example, using spin columns) or gravity. In some examples, the sample may be processed to reduce or remove cells from the sample, for example by centrifugation, either prior to or after addition of solvents, buffers, or other additives, or other processing steps (such as sample concentration).

Exemplary metabolites of use in the disclosed methods (individually or in any combination of two or more) include those shown in Tables 1, 2, and 5-8. In some examples, the metabolites are defined as mass/charge (m/z) features. Mass spectrometry data along with retention times by chromatography provide unique identifiers of these features, even though their specific chemical identities may not yet have been determined. Thus, in some examples, the metabolites of use in the disclosed methods are identified by m/z and/or retention time data, rather than by molecular name.

TABLE 1
Metabolites in cell culture supernatant during Plasmodium infection
Metabolites in Cell Culture Supernatant

3-methylindole
Succinylacetone
S-methyl-L-thiocitrulline
O-arachidonoyl glycidol
Arginine
Dioleoylphosphatidylcholine
Linoleic acid
Glycylproline
Phosphorylcholine
Sphingomyelin
Indoleacrylic acid
Isoleucine
Leucine
Thiamine
Leu-Val-OH
Thr-Val-OH
Val-Tyr-OH
Thr-Phe-OH
Dihydroxy-oxacholecalciferol
Leukotriene E4 (LTE4)
Hydroxyerythynone
Phosphatidic acids
Phosphatidylcholine
Phosphatidylethanolamine
Phosphatidylserine
Phosphatidylglycerol
Diacylglycerol
Methylimidazole
Eicosatrenoic acid
Pro-Ser-Val
Val-Thr-Thr
Gln-Phe-Met
Glu-Trp-Met
Arg-Ile-Tyr
Leu-Tyr-Arg
Asn-Gly-Lys
Ala-Thr
Gly-Pro
m/z 115.99
m/z 116.00
m/z 132.00
m/z 133.10
m/z 140.07
m/z 144.98
m/z 146.98
m/z 152.04
m/z 173.03
m/z181.01
m/z183.01
m/z 191.04
m/z 205.02
m/z 205.96
m/z 209.07
m/z 222.10
m/z 222.888
m/z 231.97
m/z 232.09
m/z 263.92
m/z 301.04
m/z 493.00
m/z 502.37
m/z 504.83
m/z 546.40
m/z 546.87
m/z 560.99
m/z 590.43
m/z 612.14
m/z 627.11
m/z 669.10
m/z 680.13
m/z 719.03
m/z 737.08
m/z 819.21

TABLE 2
Metabolites in cell pellet during Plasmodium infection
Metabolites in Cell Pellet

Arginine
Phosphatidylcholine
Lysophosphatidylcholine
Sphingosine
Palmitoylethanolamide
12-amino-octadecanoic acid
m/z 248.16
m/z 258.89
m/z 301.29
m/z 346.73
m/z 372.35
m/z 468.30
m/z 495.33

The disclosed methods include detecting the presence of one or more (such as 1, 2, 3, 4, 5, 6, 7, 8, 9, 10, 15, 20, 25, 50, 75, or more) of the disclosed metabolites, for example those in Table 1 and/or 2. In some examples, the method includes detecting presence of all of the metabolites in Table 1 and/or all of the metabolites in Table 2. In other, non-limiting examples, the one or more metabolites are not 3-methylindole, succinylacetone, S-methyl-L-thiocitrulline, O-arachidonoyl glycidol, isoleucine, arginine, or phosphorylcholine. In some embodiments, the one or more metabolites include at least one (such as 1, 2, 3, 4, 5, 6, or all) of 3-methylindole, succinylacetone, S-methyl-L-thiocitrulline, O-arachidonoyl glycidol, isoleucine, arginine, and phosphorylcholine. In some examples, the one or more metabolites in Table 1 and/or Table 2 are detected in a blood, plasma, serum, saliva, urine, or sweat sample from a subject.

In other embodiments, the disclosed methods include detecting the presence of one or more (such as 1, 2, 3, 4, 5, 6, 7, 8, 9, or 10) of the metabolites in any one of Tables 5-8. In some examples, the method includes detecting presence of each of the metabolites in Table 5, each of the metabolites in Table 6, each of the metabolites in Table 7, and/or each of the metabolites in Table 8. In some examples, the one or more metabolites in Table 1 and/or Table 2 are detected in a blood, plasma, serum, saliva, urine, or sweat sample from a subject. In particular examples, the methods include detecting presence of one or more (such as 1, 2, 3, 4, 5, 6, 7, 8, 9, 10, or more, or all) of the metabolites in Table 5 and/or Table 6 in a saliva sample from a subject. In other particular examples, the methods include detecting presence of one or more (such as 1, 2, 3, 4, 5, 6, 7, 8, 9, 10, or more, or all) of the metabolites in Table 7 and/or Table 8 in a urine sample from a subject.

In one example, the methods include detecting presence of one or more (such as 1, 2, 3, 4, 5, 6, 7, 8, 9, or 10) metabolites selected from metabolites identified in saliva samples of malaria-infected subjects using Q-Exactive HF (High-Field) Hybrid Quadrupole-Orbitrap Mass Analyzer Mass Spectrometer (HF-QE; Thermo Scientific, Waltham, Mass.) coupled with C18 liquid chromatography and having an m/z of 386.707724 and retention time of 65 seconds, an m/z of 604.419416 and a retention time of 111 seconds (such as rhodoxanthin or cholesterol glucuronide), an m/z of 108.517689 and a retention time of 244 seconds, an m/z of 286.043791 and a retention time of 119 seconds (such as ticlopidine, flamprop and/or flamprop-M, clopidogrel, cyanofenphos, moxonidine, trans-2-(4-nitrophenyl)-3-phenyl-oxirane, 1,3-dihydroxy-N-methylacridone, N-benzoylanthranilate, 7-ethoxyresorufin, mukonidine, or koeniginequinone A), an m/z of 227.0114 and a retention time of 24 seconds (such as CCCP, aluminum acetate, dehydro-4-methoxycyclobrassinin, or 5H-pyrrolo[3,4-b]pyrazin-5-one, 6-(5-chloro-2-pyridinyl)-6,7-dihydro-7-hydroxy), an m/z of 465.04142 and a retention time of 87 seconds (such as piretanide sulfate or 0-desmethyltolrestat sulfate), an m/z of 874.64237 and a retention time of 221 seconds (such as C24 sulfatide), an m/z of 529.865684 and a retention time of 104 seconds, an m/z of 758.009261 and a retention time of 133 seconds, and an m/z of 85.0285911 and a retention time of 91 seconds (such as 3-; 4-hydroxy-2-butynal, 2(5H)-furanone, 2(3H)-furanone, succinic acid semialdehyde, acetoacetic acid, 3-methyl pyruvic acid, 2-methyl-3-oxopropanoic acid, 4-hydroxycrotonic acid, (S)-methylmalonic acid semialdehyde, 2-methyl-3-oxo-propanoic acid, or acetic anhydride).

In another example, the methods include detecting presence of one or more (such as 1, 2, 3, 4, 5, 6, 7, 8, 9, or 10) metabolites selected from metabolites identified in saliva samples of malaria-infected subjects using Q-Exactive HF (High-Field) Hybrid Quadrupole-Orbitrap Mass Analyzer Mass Spectrometer (HF-QE; Thermo Scientific, Waltham, Mass.) coupled with C18 liquid chromatography and having an m/z of 1122.678802 and a retention time of 107 seconds, an m/z of 583.866644 and a retention time of 101 seconds, an m/z of 112.0107116 and a retention time of 143 seconds, an m/z of 427.7743432 and a retention time of 119 seconds, an m/z of 723.3652248 and a retention time of 117 seconds (such as marshdimerin), an m/z of 1030.147415 and a retention time of 66 seconds, an m/z of 952.6397276 and a retention time of 98 seconds, an m/z of 924.3574519 and a retention time of 67 seconds, an m/z of 524.4171783 and a retention time of 23 seconds, and an m/z of 963.1861344 and a retention time of 67 seconds (such as 2-naphthoyl-CoA).

In an additional example, the methods include detecting presence of one or more (such as 1, 2, 3, 4, 5, 6, 7, 8, 9, or 10) metabolites selected from metabolites identified in urine samples of malaria-infected subjects using Q-Exactive HF (High-Field) Hybrid Quadrupole-Orbitrap Mass Analyzer Mass Spectrometer (HF-QE; Thermo Scientific, Waltham, Mass.) coupled with C18 liquid chromatography and having an m/z of 203.99138 and a retention time of 258 seconds (such as 2-methylthiobenzothiazole), an m/z of 305.16444 and a retention time of 66 seconds (such as 10-hydroxy desipramin, 2-hydroxydesmethyl imipramine, AG-17, yohimbic acid, 3-hydroxyquinine, 11-hydroxytubotaiwine, 2′-oxoquinidine, quinine-N′-oxide, 3-hydroxyquinidine, quinidine N′-oxide, quinine 10,11-epoxide, quinine-N-oxide, 4-hydroxy nonenal mercapturic acid-d3, PtdIns-(1-arachidonoyl-d8, 2-arachidonoyl), akuammicine, gelsemine, gardneral, quinidinone, 4-chlorotestosterone, 16beta-chloro-17beta-hydroxyandrost-4-en-3-one, 19-chloro-17beta-hydroxyandrost-4-en-3-one, (E,E)-lansamide 1, girinimbine, or lansium amide B), an m/z of 214.08772 and a retention time of 63 seconds (such as benzyl nicotinate, fenamic acid, salicylanilide, 2-(4-methyl-5-thiazolyl)ethyl butanoate, 2-(4-methyl-5-thiazolyl) ethyl isobutyrate, trihomomethionine, menadione, 1-naphthoic acid, vitamin K3, dehyromatricaria ester, (Z)-2-decene-4,6,8-triynoic acid methyl ester, 1-hydroxy-2-naphthaldehyde, 2-naphthoic acid, 3Z-undecene-5,7,10-triynoic acid, or 4E-undecene-6,8,10-triynoic acid), an m/z of 179.04712 and a retention time of 277 seconds (such as L-Cys-Gly, cysteinyl-glycine, glycyl-cysteine, 1-naphthaldehyde, 2-naphthaldehyde, (S)-ACPA, (+/−)-3-(ethylthio) butanol, 2-mercapto-2-methyl-1-pentanol, (+/−)-4-mercapto-4-methyl-2-pentanol, 3-mercapto-2-methyl pentanol, 4-methoxy-2-methyl-2-butane thiol, or 3-mercapto-1-hexanol), an m/z of 387.92813 and a retention time of 138 seconds, an m/z of 117.95964 and a retention time of 268 seconds, an m/z of 338.08648 and a retention time of 73 seconds (such as 2,8-dihydroxyquinoline-beta-D-glucuronide, flusilazole, DIMBOA-glucoside, DIMBOA-Glc, 2,5-diamino-6-(5-phospho-D-ribityl amino)pyrimidin-4(3H)-one, N-acetyl djenkolic acid, disulfiram, phaseolic acid, mono-trans-p-coumaroylmesotartaric acid, 2-O-feruloyltartronic acid, cis-coutaric acid, glutamyl-phenylalanine, phenylalanyl-glutamate, N,N′-(acridine-3,6-diyl)diacetamide, GYM 52466, 7-acetamidonitrazepam, or desethylenenorfloxacin), an m/z of 682.79589 and a retention time of 117 seconds, an m/z of 113.05889 and a retention time of 52 seconds (such as sorbic acid, aleprolic acid, 3,5-hexadienoic acid, 5-hexynoic acid, trans-1,2-dihydrobenzene-1,2-diol, 4-oxo-2E-hexenal, 4-oxo-2Z-hexenal, cyclohexane-1,3-dione, cyclohexane-1,2-dione, 1,4-cyclohexanedione, parasorbic acid, C6:2n-1,3, 3-hexynoic acid, 4-hexynoic acid, 5-hexynoic acid, 5-hexyn-1-oic acid, 2-hexenedial, 3-hexenedial, 6-hydroxy-2,4-hexadienal, 5,5-dimethyl-2(5H)-furanone, 2,5-dimethyl-3(2H)-furanone, syoyualdehyde, 2-(methoxymethyl)furan, xi-3,5-dimethyl-2(5H)-furanone, 5,5-dimethyl-2(5H)-furanone, or 2-hydroxy-3-methyl-2-cyclopenten-one), or an m/z of 726.72644 and a retention time of 111 seconds.

In a further example, the methods include detecting presence of one or more (such as 1, 2, 3, 4, 5, 6, 7, or 8) metabolites selected from metabolites identified in urine samples of malaria-infected subjects using Q-Exactive HF (High-Field) Hybrid Quadrupole-Orbitrap Mass Analyzer Mass Spectrometer (HF-QE; Thermo Scientific, Waltham, Mass.) coupled with C18 liquid chromatography and having an m/z of 692.3898116 and a retention time of 26 seconds (such as fumonisin C2 or fumonisin C3), an m/z of 271.9162641 and a retention time of 179 seconds, an m/z of 288.1305573 and a retention time of 85 seconds (such as naftifine hydrochloride, asparginyl-asparagine, N2-oxalyl arginine, lysyl-proline, or prolyl-lysine), an m/z of 954.3185803 and a retention time of 67 seconds, an m/z of 352.0259716 and a retention time of 66 seconds (such as oxine-copper, iprodione, or dCMP), an m/z of 260.0110747 and a retention time of 165 seconds (such as riluzolamide), an m/z of 646.7783966 and a retention time of 73 seconds, or an m/z of 674.4584899 and a retention time of 73 seconds (such as PE(14:0/20:5(5Z,8Z,11Z,14Z,17Z)), PE(14:1(9Z)/20:4(5Z,8Z,11Z,14Z)), PE(14:1(9Z)/20:4(8Z,11Z,14Z,17Z)), PE(16:1(9Z)/18:4(6Z,9Z,12Z,15Z)), PE(18:4(6Z,9Z,12Z,15Z)/16:1(9Z)), PE(20:4(5Z, 8Z,11Z,14Z)/14:1(9Z)), PE(20:4(8Z,11Z,14Z,17Z)/14:1(9Z)), or PE20:5(5Z,8Z,11Z,14Z,17Z)/14:0)).

In some examples, the methods also include comparing amounts of the one or more metabolites in the sample to a control and identifying the presence of Plasmodium infection in the sample (or the subject) if there is a change in amount (such as an increase or a decrease, for example a statistically significant increase or decrease) of the one or more metabolites in the sample as compared to the control. The control can be any suitable control against which to compare an amount of one or more metabolites (such as one or more of the metabolites disclosed in any of Tables 1, 2, and 5-8) in a sample from a subject. In some embodiments, the control sample is a sample from a subject known not to be infected with Plasmodium or a pool of samples from subjects known not to be infected with Plasmodium. In other embodiments, the control is a reference value or ranges of values. For example, the reference value can be derived from the average metabolite values obtained from a group of non-Plasmodium infected control subjects. In some examples, the control includes a level of metabolites of a signature (such as normalized or aggregate values) from a control or reference dataset (such as metabolic profile data from one or more samples from non-Plasmodium infected subjects). In other examples, a control is a threshold level or amount of a biomarker that indicates a presence of a condition. In particular embodiments, the control sample is a sample of the same type (e.g., blood, saliva, or urine) as the experimental sample or is a reference value, threshold, or cutoff derived from samples of the same type of sample as the experimental sample.

In some examples, an increase of at least about 1.2-fold (such as at least about 1.5-fold, at least about 2-fold, at least about 3-fold, at least about 5-fold, at least about 10-fold, at least about 20-fold, at least about 50-fold, at least about 100-fold, at least about 500-fold, at least about 1000-fold, or more) compared to a control indicates presence of Plasmodium in the sample from the subject and/or indicates that the subject from which the sample was obtained is infected with Plasmodium. In other examples, a decrease of at least about 10% (such as at least about 15%, at least about 20%, at least about 25%, at least about 30%, at least about 40%, at least about 50%, at least about 60%, at least about 70%, at least about 80%, at least about 90%, at least about 95%, or more) compared to a control indicates presence of Plasmodium in the sample from the subject and/or indicates that the subject from which the sample was obtained is infected with Plasmodium. In particular examples, an increase in one or more of 3-methylindole, succinylacetone, S-methyl-L-thiocitrulline, or O-arachidonoyl glycidol compared to a control indicates presence of Plasmodium in the sample or the subject. In other particular examples, a decrease in isoleucine and/or arginine compared to a control indicates presence of Plasmodium in the sample or the subject.

Presence of the disclosed metabolites can be detected using any suitable means known in the art. For example, detection of metabolites can be accomplished by mass spectrometry methods, immunoassays, aptamer binding, and/or chromatographic methods. Additional methods of detecting small molecules, such as the metabolites disclosed herein, are well known in the art, and are discussed in more detail below.

In some embodiments, an amount of one or more of the disclosed metabolites (such as 1, 2, 3, 4, 5, 6, 7, 8, 9, 10 or more, or all of the metabolites in any of Tables 1, 2, and 5-8 or a combination thereof) is detected in a sample and the amount of each is normalized relative to one or more metabolites in the same sample. In some examples, an aggregate value that is obtained by calculating the amount of each of the metabolites in a signature (such as one or more of the those shown in Tables 1, 2, and 5-8) and using a positive (+1) or negative (−1) weighting for each metabolite depending on whether it is positively or negatively regulated by Plasmodium infection is calculated. In some examples, normalized amount of the metabolite(s) (or normalized amount of the metabolomic signature) or an aggregate value is determined to be increased or decreased as compared to median normalized amount of the metabolite (or signature) or an aggregate value for a set of samples. In some examples, the median normalized amount or aggregate value is obtained from publicly available metabolomic datasets.

In some embodiments, the disclosed methods further include administering to a subject (such as a subject from which a sample having presence of Plasmodium was obtained) a therapeutically effective amount of one or more agents for treating malaria. In some embodiments, the therapeutic agent is artemisinin or a derivative thereof or a quinolone-based compound. In some examples, the therapeutic agent is artemisinin or a derivative thereof (such as artesunate, dihydroartemisinin, or artemether) or an artemisinin-based combination therapy (such as artemether-lumefantrine), atovaquone-proguanil, chloroquine, primaquine, mefloquine, quinine (alone or with doxycycline, tetracycline, or clindamycin), or a combination of two or more thereof. Appropriate treatment options can be selected by a skilled clinician, for example, based on the Plasmodium species prevalent in the geographic area of the subject (or of the subject at the time of exposure), known drug-resistance of the Plasmodium prevalent in the area, age and overall health status of the subject, and other factors.

### III. Methods of Detecting Metabolites

As described herein, the amount of one or more metabolites in a sample can be detected using any one of a number of methods well known in the art. Although exemplary methods are provided, the disclosure is not limited to such methods, and include other methods known to one of skill in the art, such as detection of product(s) of a chemical reaction or an enzyme-catalyzed reaction or an electronic sensor (e.g., an “electronic nose”) to detect volatile compounds (such as 3-methylindole).

In some embodiments, the amount of one or more of the metabolites disclosed herein is detected using mass spectrometry (MS), tandem mass spectrometry (MS/MS), or mass spectrometry in combination with liquid chromatography (LC-MS or LC-MS/MS) or gas chromatography (GC-MS or GC-MS/MS). In particular embodiments, the one or more metabolites are detected with linear quadrupole ion trap (LTQ)-Fourier transform mass spectrometer (FTMS) coupled with liquid chromatography (such as C18 liquid chromatography). In other particular embodiments, the one or more metabolites are detected with high-field hybrid quadrupole mass spectrometry coupled with liquid chromatography (such as C18 liquid chromatography). In other examples, the one or more metabolites are detected with liquid chromatography-electrospray ionization mass spectrometry (LC-ESI-MS or LC-ESI-MS/MS), LC-atmospheric pressure chemical ionization (APCI) mass spectrometry (LC-APCI-MS), LC-quadrupole time of flight (TOF) mass spectrometry, multipass TOF-MS, matrix-assisted laser desorption/ionization (MALDI)-TOF-MS, or MALDI-LTQ-MS. Particular examples of mass spectrometry methods for detecting metabolites are described in Examples 1 and 3, below.

In other embodiments, the amount of one or more of the metabolites disclosed herein is detected by one of a number of immunoassay methods that are well known in the art, such as those presented in Harlow and Lane (Antibodies, A Laboratory Manual, CSHL, New York, 1988). Methods of constructing antibodies specific for the disclosed metabolites are known in the art. In some examples, antibodies to metabolites described herein are known (e.g., Tuomola et al., J. Immunol. Meth. 240:111-124, 2000 (3-methylindole)). Other antibodies can be obtained from commercial sources (e.g., Abcam, Cambridge, Mass.; Santa Cruz Biotechnology, Dallas, Tex.; and others) or can be prepared by one of skill in the art.

Any standard immunoassay format can be used to measure metabolite levels. Exemplary immunoassays include, but are not limited to Western blotting, ELISA, radioimmunoassay, fluorescence microscopy, and flow cytometry. Thus, in one example, levels of one or more of the metabolites listed in Tables 1, 2, and 5-8 can readily be evaluated using these methods. Immunohistochemical techniques can also be utilized for metabolite detection. General guidance regarding such techniques can be found in Bancroft and Stevens (Theory and Practice of Histological Techniques, Churchill Livingstone, 1982) and Ausubel et al. (Current Protocols in Molecular Biology, John Wiley & Sons, New York, 1998).

In other embodiments, the amount of one or more of the metabolites disclosed herein is detected by an aptamer-based assay. Aptamers are small nucleic acid molecules (e.g., RNA or DNA) or peptides that bind to a target molecule (such as a metabolite disclosed herein) with high affinity and specificity. Aptamer specificity for a target is based on its tertiary structure, which is determined by its primary sequence and by hydrophobic and ionic interactions with the target. One of skill in the art can develop aptamers for a target molecule, using methods such as SELEx (Systematic Evolution of Ligands by Exponential enrichment). See, e.g., Ellington et al., Nature 346:818-822, 1990; Turek et al., Science 249:505-510, 1990; Stoltenburg et al., Biomol. Eng. 24:381-403, 2007; Ohuchi, Biores. Open Access 1:265-272, 2012.

Generally, the detection of one or more of the disclosed metabolites with an aptamer involves the use of molecular methods and the detection of a signal, such as fluorescent, radioactive, or enzymatic readout. Any immunoassay known to the art that utilizes a protein-based method of detecting a molecule of interest can be adapted for use in detecting the molecule with an aptamer. Such binding agents can include a detectable label (such as a radiolabel, fluorophore or enzyme), that permits detection of the binding of the aptamer to the metabolite and determination of relative or absolute quantities of the molecule of interest in the sample.

Although the details of the aptamer-based assays may vary with the particular format employed, the method of detecting a metabolite in a sample using an aptamer generally includes the steps of contacting the sample with an aptamer under conditions sufficient to form a binding complex between the aptamer and the target metabolite, and detecting the presence and/or quantity of the binding complex (bound aptamer), either directly or indirectly. Exemplary immunoassays that can be modified for use with aptamers include, but are not limited to Western blotting, ELISA, radioimmunoassay, fluorescence microscopy, and flow cytometry.

One of skill in the art can identify additional methods for use in detecting the metabolites disclosed herein. For example, metabolites can be detected using nuclear magnetic resonance (NMR) spectroscopy, infrared spectroscopy, thin layer chromatography, high performance liquid chromatography (HPLC), or gas chromatography. Voltammetry can also be used to detect metabolites in a sample (e.g., International Pat. Publ. No. WO 2005/001463).

The present disclosure is illustrated by the following non-limiting Examples.

### Example 1

### Materials and Methods—Blood Samples

Parasite culture: In this study, asynchronized culture was employed. The purpose of using asynchronized culture was to capture small metabolite molecules that might be commonly released by all stages of parasites. The laboratory-adapted 3D7 strain of Plasmodium falciparum was used. The asynchronized blood stage parasites were cultured as described (Trager et al., Science 193:673-675, 1976) in RPMI 1640 medium supplemented with 10% heat-inactivated O+ human serum, 1 μg/ml gentamicin, 36 μM hypoxanthine, 31 mM HEPES and 25 mM sodium bicarbonate. Four flasks of parasite culture with 3% hematocrit and 0.5% starting parasitemia were prepared at the same time using red blood cells from different donors. At the same time, four flasks of culture without parasite inoculation but containing the same culture medium and hematocrit were also prepared. Culture materials, including supernatants and cell pellets, from all the infected and non-infected flasks were collected at 12, 24, 36, and 48 hours without changing or adding culture medium. In total, 16 supernatant samples obtained from the infected flasks and the same numbers of supernatant samples from non-infected flasks were used for this study. All the samples used in this study were mycoplasma free. Parasite densities in the infected flasks were recorded as % count by blood smear reading. They increased with time, 0.83%±0.1%, 1.05%±0.06%, 1.45%±0.20% and 2.38%±0.30% (mean±SD) at 12, 24, 36 and 48 hours, respectively.

C18 Liquid Chromatography Coupled with Fourier-Transform Mass Spectrometry (FTMS):

All the samples were run in duplicate. Aliquots of supernatant samples (100 μl) were treated with acetonitrile (2:1, v/v), spiked with 2.5 μl internal standard mix, and centrifuged at 14,000×g for 5 minutes at 4° C. to remove protein as described previously (Johnson et al., Analyst 135:2864-2870, 2010). Then LTQ-FTMS (Thermo, hybrid linear ion trap-Fourier Transform Ion Cyclotron Resonance mass spectrometry, Waltham, Mass.) coupled with C18 liquid chromatography was run on those collected samples. HRM offers an important advantage in analysis of highly complex metabolite mixtures, such as biological extracts, because detection of mass/charge (m/z) with 5 ppm or better mass resolution and mass accuracy substantially decreases the demand for physical separation prior to detection. Detection of m/z of ions from 85 to 850 with 50,000 resolution over 10 min LC runs with data extraction using apLCMS (Yu et al., Bioinformatics 25:1930-1936, 2009) provided a minimum of 3000 reproducible features, many with sufficient mass accuracy to allow prediction of elemental composition. An m/z feature is defined by m/z, RT (retention time), and ion intensity (integrated ion intensity for the peak).

The Kyoto Encyclopedia of Genes and Genomes (KEGG) database (available on the World Wide Web at genome.jp/keg) was utilized to map the features distribution on both human and Plasmodium metabolic pathways (Kanehisa Novartis Foundation Symposium 247:91-101, 2002; Kanehisa et al., Nucl. Acids Res. 28:27-30, 2000). Examination of m/z of metabolites in the KEGG human and Plasmodium metabolomics pathways showed that less than 10% of metabolites are redundant with others in terms of elemental composition. Identified metabolites were annotated using Metlin Mass Spectrometry Database (available on the World Wide Web at metlin.scripps.edu; Smith et al., Ther. Drug Monitor. 27:747-751, 2006). Direct examination by MS/MS of selected accurate mass m/z features of human plasma showed that for many m/z, the ion dissociation patterns matched those of authentic standards with identical elution times. For such metabolites, quantification relative to stable isotope internal standards had a coefficient of variation (5 to 10%) and sensitivity (low nanomolar to picomolar) which was similar to other methods and sufficient in allowing targeted analysis of selected chemicals within the context of an information-rich non-targeted profiling of all m/z detected within biological samples.

Metabolic Profiling with Univariate and Multivariate Statistical Analysis:

Analyses were performed based on the results from both biological and technical replicates. Total features of culture supernatant were collected after processing mass spectral data with apLCMS. The features from duplicate LCMS analyses were averaged, log 2 transformed, and quantile normalized for subsequent statistical and bioinformatics analyses including univariate analysis, Manhattan plot, and false discovery rate (FDR; Benjamini et al., J.R. Statis. Soc. B B57:289-300, 1995) to determine the significant metabolites between infected and non-infected cultures. Furthermore, the metabolic profiles were discriminated using Limma-hierarchical cluster analysis to separate two groups in association with metabolites. Limma was originally a package of Linear Models for Microarray to analyze the gene expression data arising from microarray or RNA-Seq technologies from Bioconductor. A core capability of this study was the use of linear models to assess differential expression in the context of multifactor designed experiments Limma provided the ability to make comparisons between many targets simultaneously including metabolites (Cribb et al., AIDS Res. Hum. Retroviruses 30:579-585, 2014; Neujahr et al., Am J. Transplant. 14:841-848, 2014).

Pathway Analysis with KEGG:

The database of Kyoto Encyclopedia of Genes and Genomes (KEGG) was utilized to map the features distribution on both human and Plasmodium metabolic pathways. Detected m/z features matching known human and Plasmodium intermediary metabolites were mapped to a pathway map; most human and known Plasmodium metabolic pathways were represented.

Quantification of 3-Methylindole and Succinylacetone:

3-methylindole (M51458) and succinylacetone (D1415) were purchased from Sigma-Aldrich (MO, USA). A standard curve was made by known concentrations (0.1-0.5 nmole) of the reagent grade compounds in cell culture media. Areas from the samples were plotted against concentrations and a standard curve with an r²>0.99. 3-methylindole and succinylacetone in supernatants from parasite culture were treated using 100 μl in microcentrifuge tubes. These tubes were centrifuged at 14000×g at 4° C. for 5 minutes. Supernatant (10 μl) was injected in mass spectrometry and resulting areas were noted. Concentrations of the compounds were calculated using the standard curve.

### Example 2

### Metabolomic Analysis

Metabolome-Wide Association Study (MWAS):

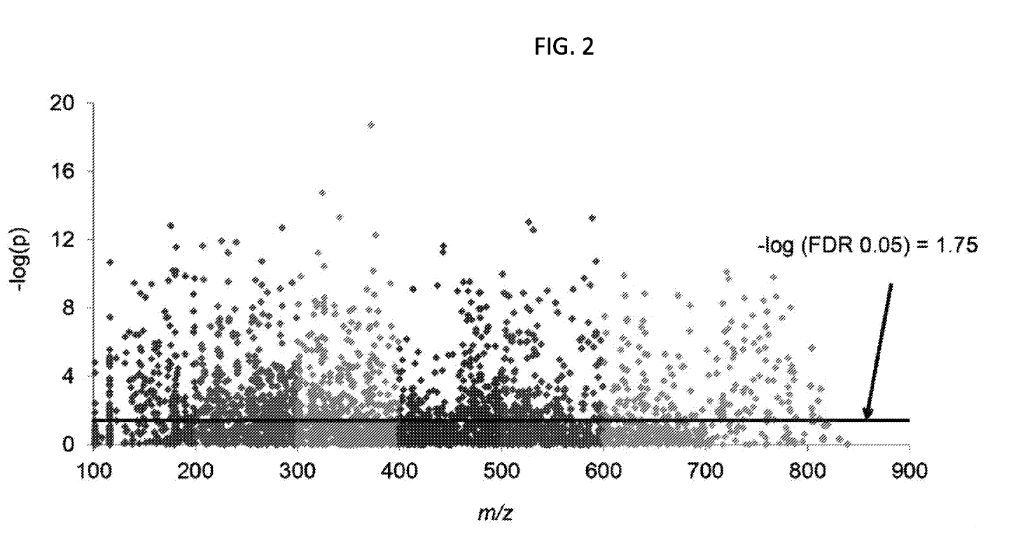

MWAS was used to identify changes in supernatants from non-infected and Plasmodium-infected cultures at all points (12, 24, 36, and 48 hours). A Manhattan plot, which combines a statistical test (e.g., p-value, ANOVA) with the magnitude of change and enables visual identification of statistically significant data-points (metabolites) that display large-magnitude changes is shown in FIG. 2. Multiple testing corrections like FDR adjusts p-values (q-values) derived from multiple statistical tests to correct for the occurrence of false positives. The Y axis represents the −log₁₀ of the raw p-value comparing supernatants of culture system between non-infected and Plasmodium-infected red blood cells. The X axis indicated m/z ranging 85-850 m/z. The dotted line was shown as the FDR significant level, therefore any m/z above this line were significantly different between two groups at FDR q=0.05. The total number of significant features was 1025 out of the 3270 detected.

Mapping Significant Features Through KEGG Human and Plasmodium Metabolic Pathways:

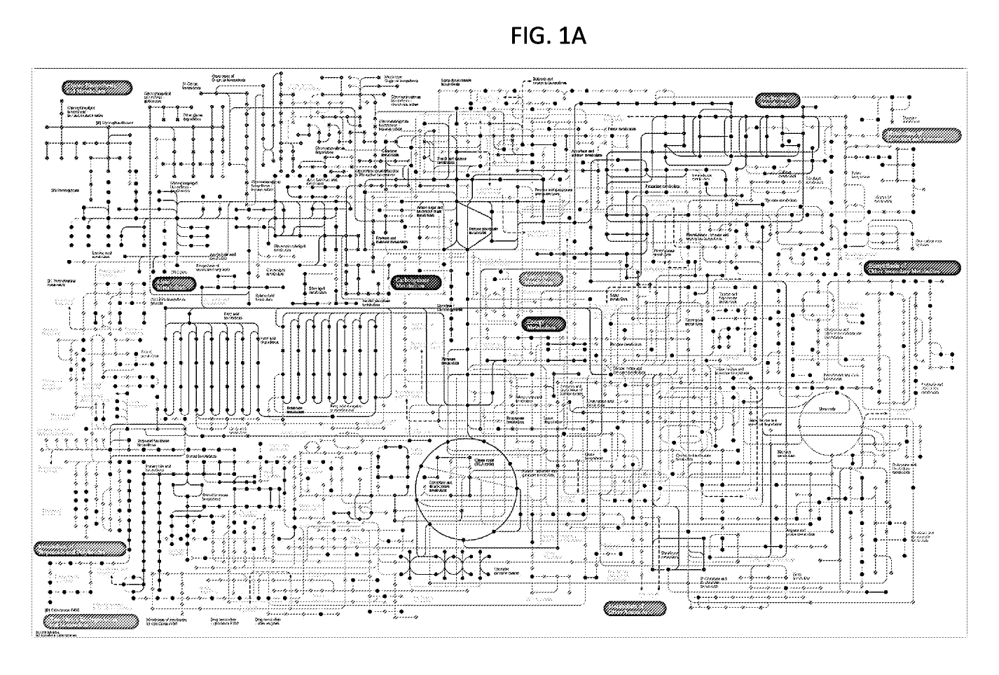

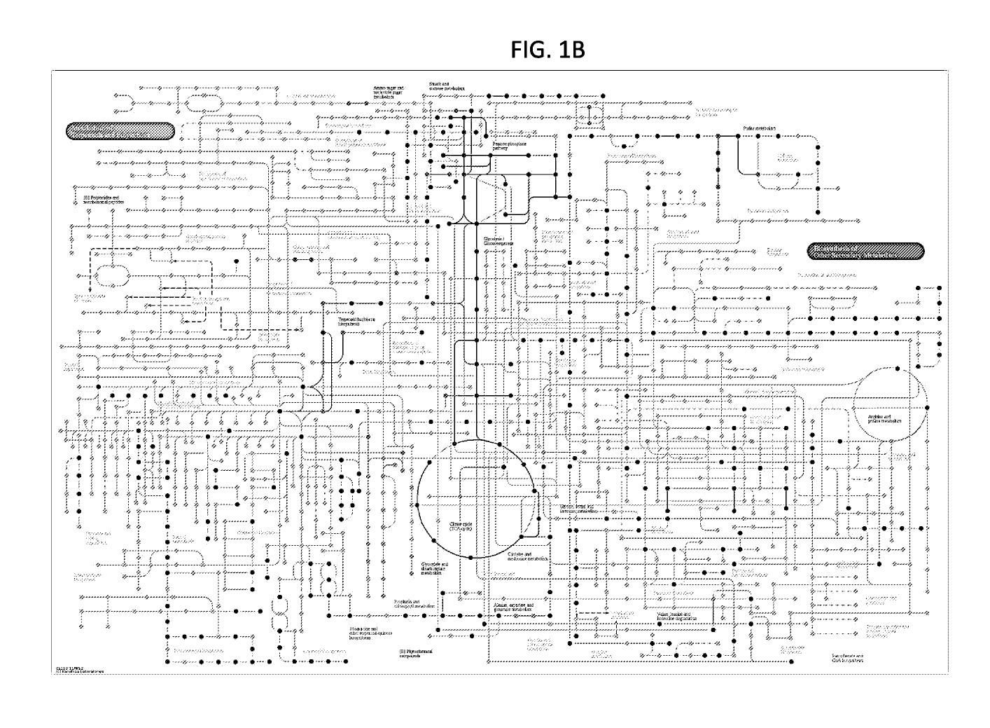

The HRM platform provides precise metabolic phenotypes to determine the possible pathways altered by Plasmodium falciparum. The schematic representations in FIGS. 1A and 1B show mapping through KEGG to both human and Plasmodium metabolic pathways. Results showed that these metabolites matched 439 metabolic compounds in both human and Plasmodium metabolic pathways. The remaining 586 of 1025 chemicals which were not matched might be either waste products of the parasite or could be utilized by unidentified Plasmodium pathways.

Two Way Hierarchical Cluster Analysis (HCA) on FDR Significant Features in Supernatant Between Non-Infected and Infected Cultures:

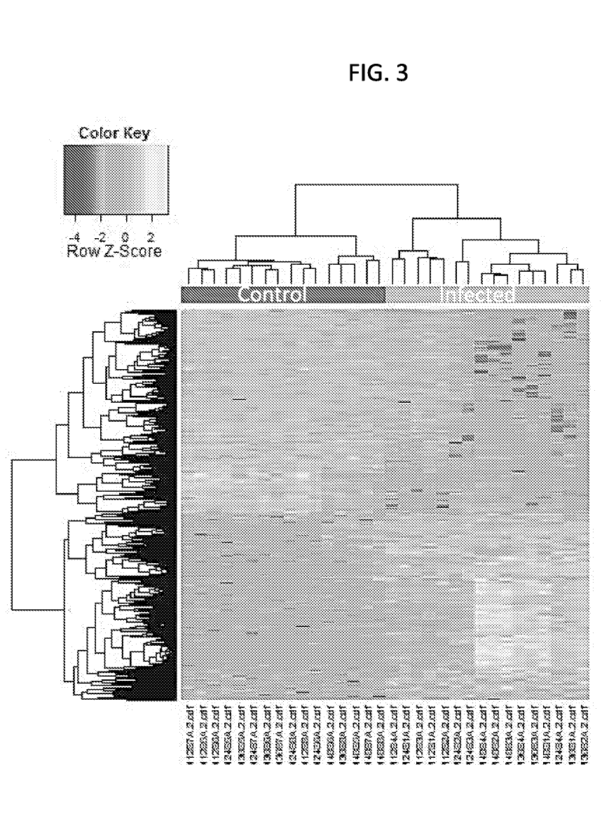

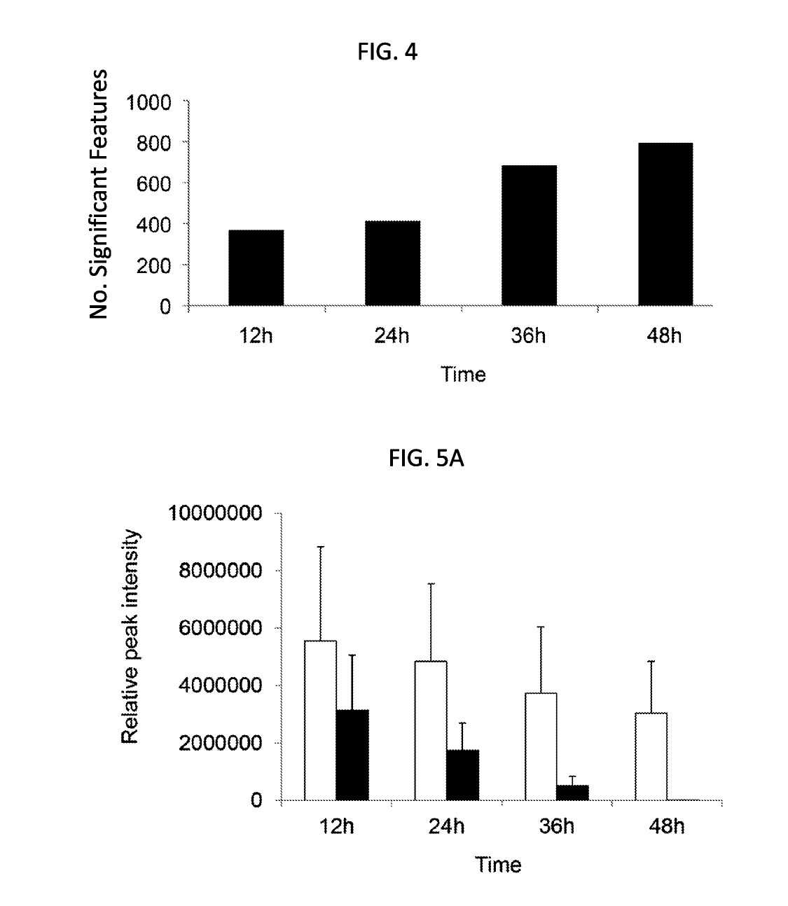

Two way HCA was performed on combined sample classification with metabolites clustering to identify which metabolites were the most important for sample grouping. In this study, HCA was performed using 1025 metabolites at FDR q=0.05, which were the key components to separate the two groups using all four time points (FIG. 3). HCA determines similarity measures using Euclidean distance and Pearson linear correlation. The top panel showed that two main clusters separate supernatant of non-infected from infected cultures. The sample name was listed at the bottom panel. The right panel included 1025 metabolites which contributed to discrimination of samples according to malaria infection. In addition, FIG. 4 shows a broad increasing trend in significant features at FDR q=0.05 during 48 hours.

Decrease in Arginine and Isoleucine:

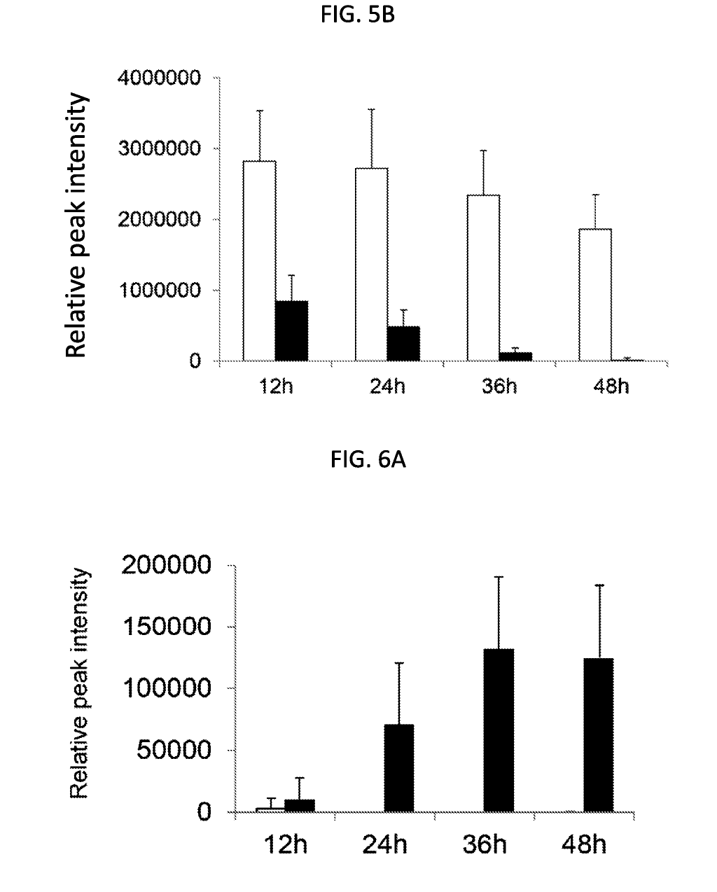

In order to validate methodology and analytical approach used for identification of the four molecules described above, the intensity of arginine and isoleucine, which are known to be consumed and critical for malaria parasite growth, were analyzed. As parasites grow within host red blood cells, they utilize large quantities of amino acids, most of which are obtained from proteolyzed hemoglobin in the host blood cells (Istvan et al., Proc. Natl. Acad. Sci. USA 108:1627-1632, 2011). In FIG. 5A, arginine decreased to zero in a time dependent fashion during 48 hours. However, hemoglobin lacks one important amino acid, isoleucine, and the parasite therefore has to source this from culture system. In FIG. 5B, isoleucine was reduced significantly in supernatants after the 48 hour incubation period.

Identification of the Potential Biomarkers Increased with Culture Time:

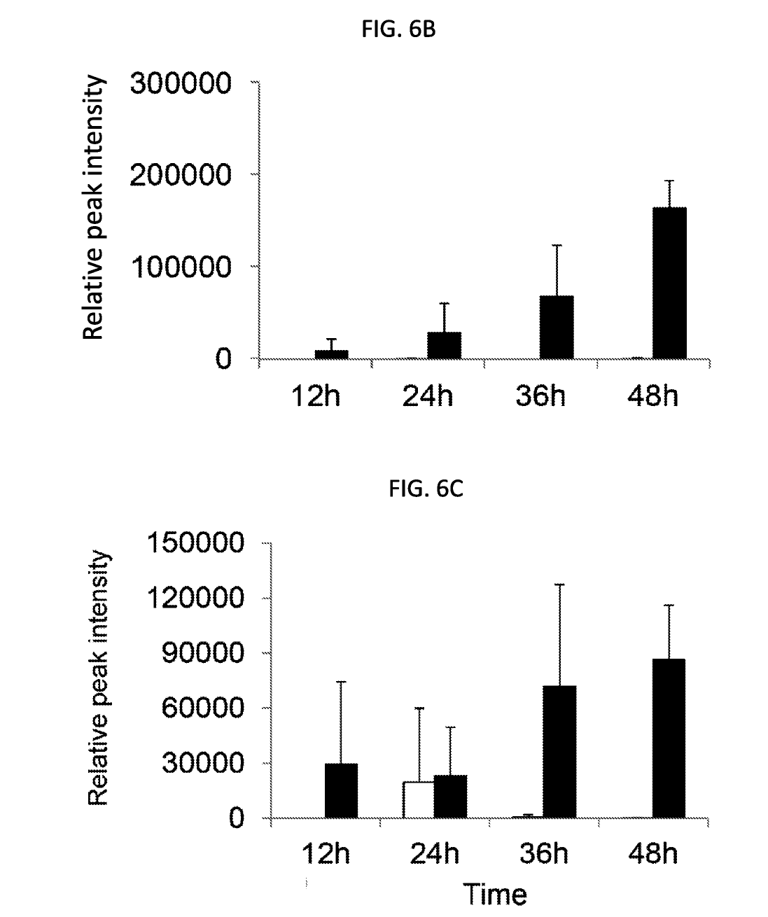

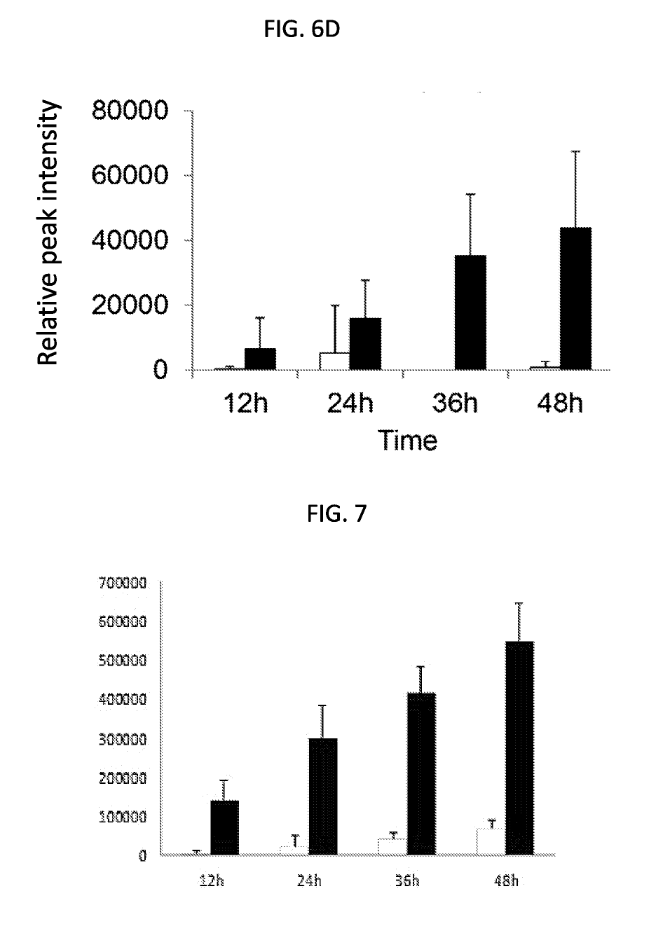

Among the remaining 586 of 1025 significant metabolites (FDR q=0.05) determined by FDR, the ion intensities of several metabolites were increased with culture time in infected culture supernatants but not in non-infected culture supernatants (Tables 1 and 2), suggesting a positive association between the quantity of these molecules released and level of parasitemia. These metabolites included 3-methylindole (FIG. 6A), succinylacetone (FIG. 6B), S-methyl-L-thiocitrulline (FIG. 6C), and O-arachidonoyl glycidol (FIG. 6D). The compound 3-methylindole has been shown to stimulate an odorant receptor to attract malaria mosquito vector (Xu et al., Biochem. Biophys. Res. Commun. 435:477-482, 2013), while succinylacetone has been identified as an inhibitor of heme biosynthesis (Ebert et al., Biochem. Biophys. Res. Commun. 88:1382-1390, 1979; Tschudy et al., J. Biol. Chem. 256:9915-9923, 1981). S-methyl-L-thiocitrulline has been identified as a potent nitric oxide synthase (NOS) inhibitor to reduce nitric oxide production and endothelial dysfunction (Bradshaw et al., Vitam. Horm. 81:191-205, 2009). Finally, O-arachidonoyl glycidol was reported to be an inhibitor of fatty acid amide hydrolase (Bradshaw et al., BMC Biochem. 10:14, 2009; McHugh et al., BMC Neurosci. 11:44, 2010).

A similar pattern was observed for phosphorylcholine (FIG. 7), the molecule that was reported in previous study using infected erythrocytes (Teng et al., Biosci. Rep. 34:e00150, 2014) and is involved in the phosphobase methylation for phosphatidylcholine production (Saen-Oon et al., J. Biol. Chem. 289:33815-33825, 2014). The observed increase in phosphorylcholine with culture time further validated the methodology and analytical approach used for identifying the four molecules reported above.

Quantification of 3-Methylindole and Succinylacetone:

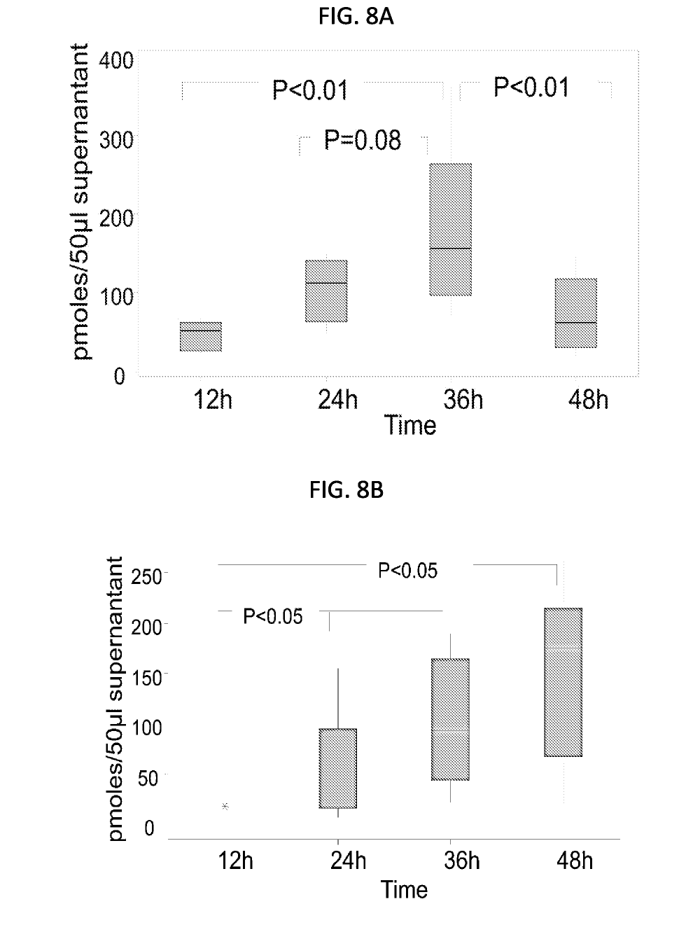

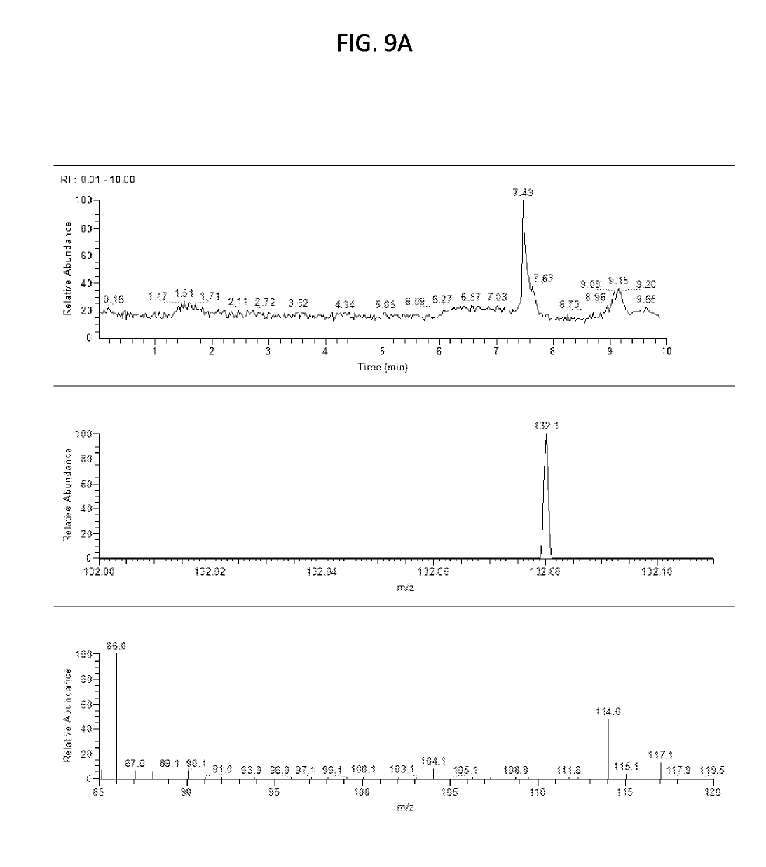

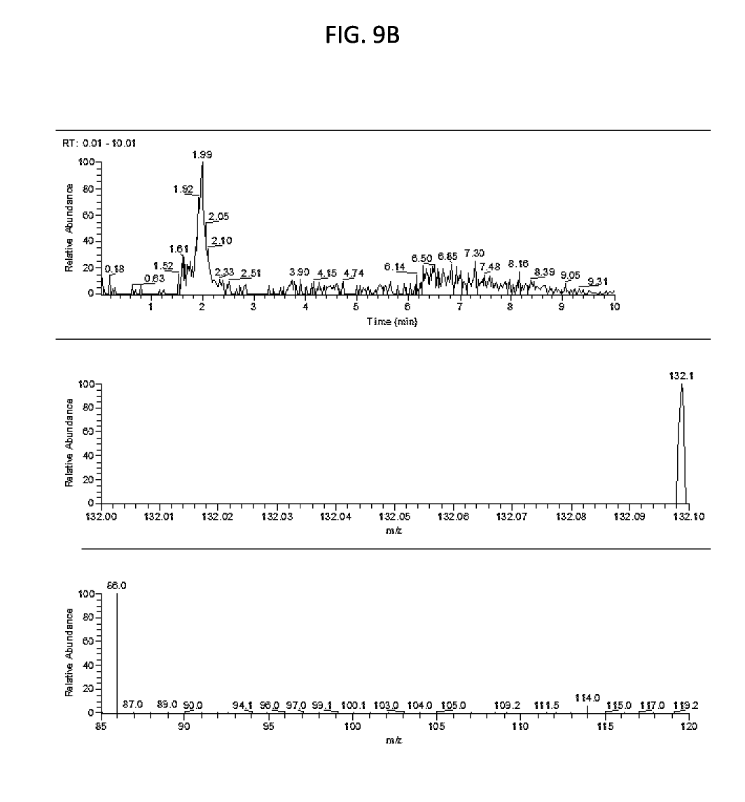

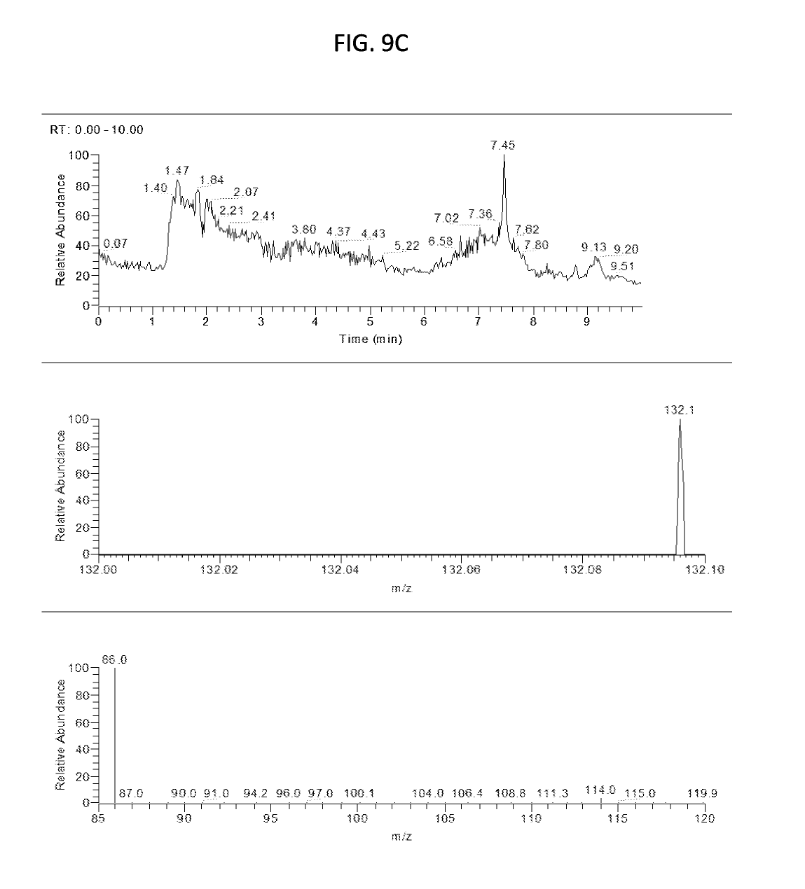

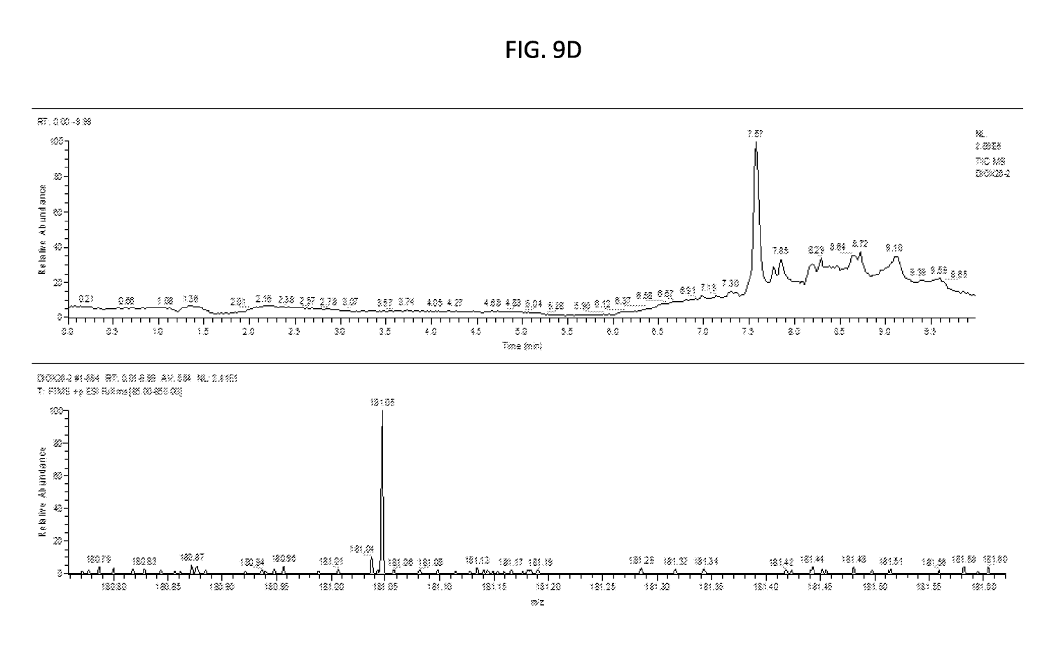

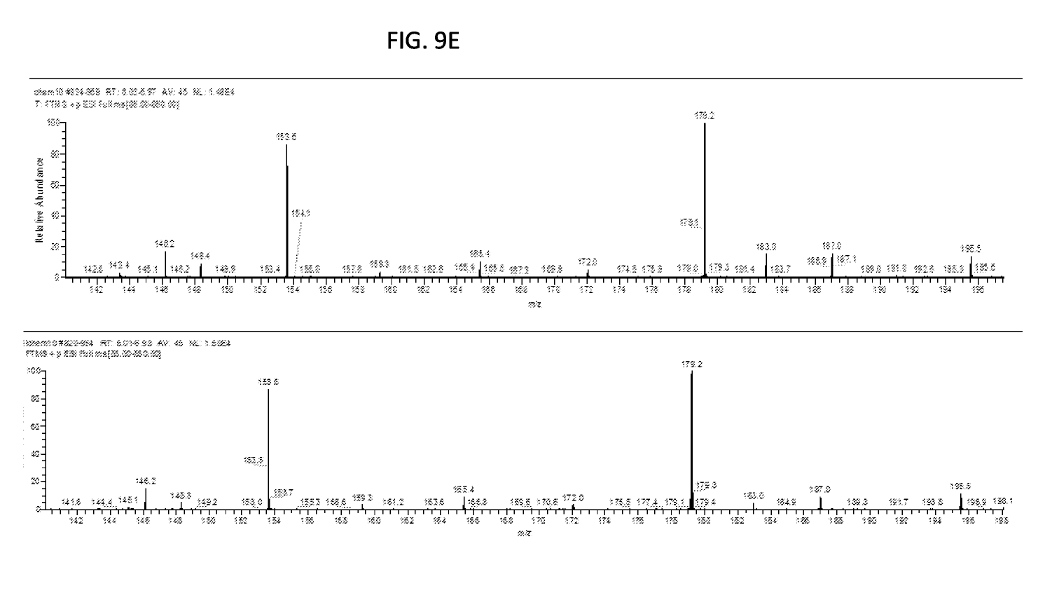

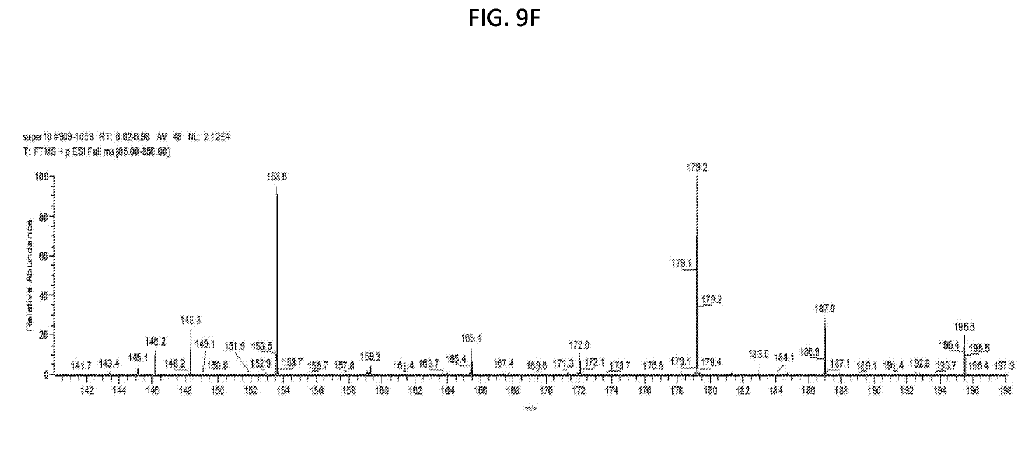

The production of 3-methylindole and succinylacetone were measured at each time point during 48 hours in 50 μl of supernatants from 3% hematocrit cultures. At 36 hours, the amount of 3-methylindole was highest and the concentration was 178±18.7 pmoles (FIG. 8A). The generation of succinylacetone increased over the time. The amounts were 2±2 pmoles at 12 hours culture, with the highest reading at 157±30.5 pmoles at 48 hours culture (FIG. 8B). Additional MS/MS data of these compounds are shown in FIGS. 9A-9F.

### Example 3

### Saliva and Urine Samples

Samples were extracted from saliva and urine of healthy and malaria-infected (P. falciparum) human subjects from Kenya. A total of 67 samples (32 healthy and 35 malaria-infected) were used. For saliva, 32 healthy and 35 malaria infected samples were analyzed, whereas for urine, 32 healthy and 31 malaria infected samples were analyzed. After sample preparation according to appropriate protocol procedures, the samples were processed with a Q-Exactive HF (High-Field) Hybrid Quadrupole-Orbitrap Mass Spectrometer (HF-QE; Thermo Scientific, MA, USA) coupled with C18 liquid chromatography, which is designed to detect over 10,000 metabolite ions per sample.

All the samples were run in triplicate. Aliquots of samples (50 μl) were treated with acetonitrile (2:1, v/v), spiked with 2.5 μl internal standard mix, and centrifuged at 13,200×g for 10 minutes at 4° C. to aliquot supernatant into vials as described previously (Johnson et al., Analyst 135:2864-2870, 2010). Then the HF-QE, coupled with C18 liquid chromatography was run on those collected samples. Detection of m/z of ions from 85 to 1275 with 120,000 resolution over 10 minute LC runs with data extraction using apLCMS (Yu et al., Bioinformatics 25:1930-1936, 2009) provided a minimum of 3000 reproducible features, many with sufficient mass accuracy to allow prediction of elemental composition.

After standard quality control and initial data filtering, two initial data groups of potential biomarkers were created:

Group 1: Initial data contained 90% of present values per metabolite across all healthy and all malaria samples. The numbers of metabolite features for downstream analysis were: 4,031 for saliva and 3,190 for urine (Table 3).

Group 2: Initial data contained combined metabolite features from (a) and (b):

(a) 35% of present values per metabolite across all control samples, and 63% of non-present values across all malaria samples, and

(b) 35% of present values per metabolite across all malaria samples, and 63% of non-present values across all control samples.

The number of metabolite features for the downstream analysis were respectively: (a) 18 for saliva and 8 for urine and (b) 11 for saliva and 3 for urine. Collectively (from (a) and (b)), there were 18+11=29 metabolite features for saliva and 8+3=11 metabolite features for urine (Table 4).

Group 1 and Group 2 of metabolites were used to maximize the biomarker discovery potential. The combined results from Method 1 and Method 2 (below) were used in both Group 1 and Group 2. The final panel of significant metabolites (putatively annotated by the METLIN database) was constructed by combining the results from Group 1 and Group 2 analyses.

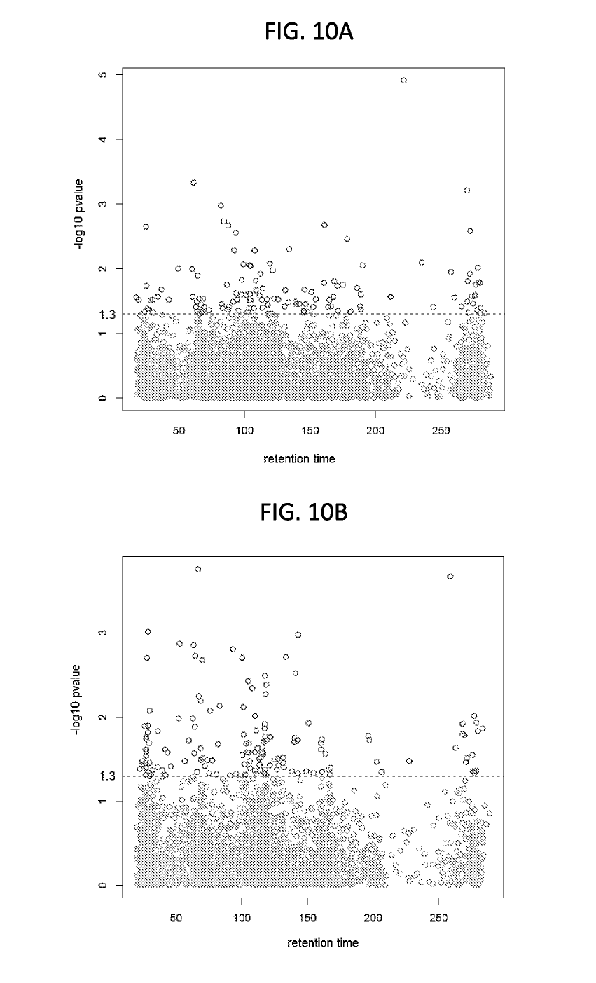

In Method 1, linear regression model was used to identify metabolite features that were significantly changed in malaria samples and were associated with parasitemia levels, controlling for age and gender. In Group 1, for saliva, the model gave 133 metabolite features, and for urine, the model gave 153 metabolite features (at the significance level of p-value<0.05, or −log 10p>1.3, FIGS. 10A and 10B and Table 3). In Group 2, for saliva, the model gave 22 metabolite features, and for urine, the model gave 8 metabolite features (Table 4).

In Method 2, Partial-Least Squares (PLS) regression model was used to identify metabolites that were highly significantly changed in malaria samples. The Variable Importance in Projection (VIP) score estimates the importance of each variable (metabolite) in the projection used in a PLS model and it is used for variable (metabolite) selection. A variable (metabolite) with a VIP Score close to or greater than 2 can be considered important in a given model. In Group 1, for saliva, the model gave 24 metabolite features, and for urine, the model gave 31 metabolite features (at a VIP score>2.5, and p-value<0.05) (Table 3). In Group 2, for saliva, the model gave 14 metabolite features, and for urine, the model gave 9 metabolite features (at a VIP score>2.5, and p-value<0.05) (Table 4).

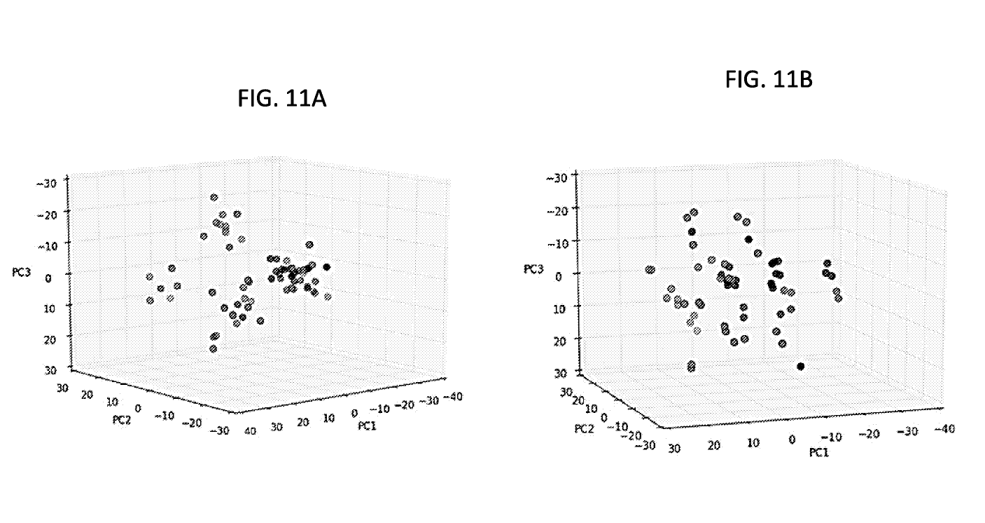

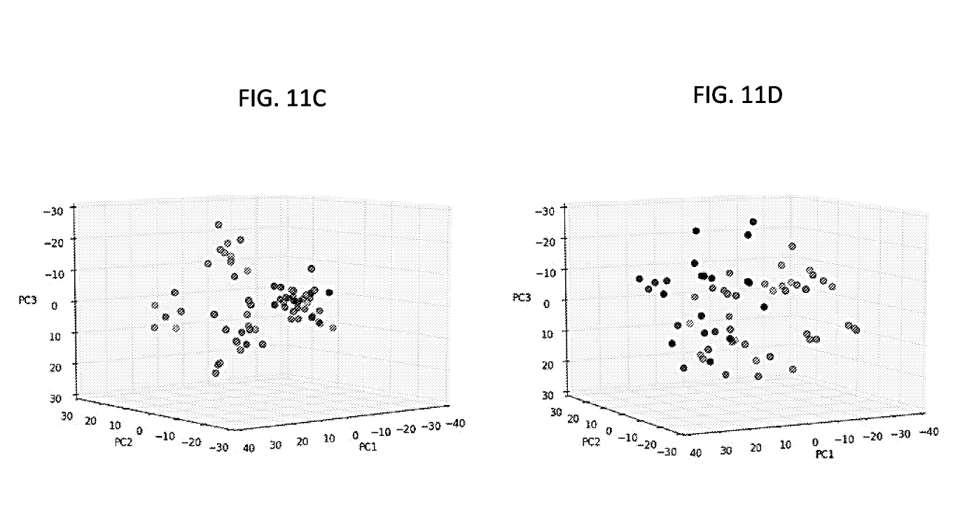

The metabolite features that passed the criteria from Model 1 and Model 2 were selected. This combinatorial methodology (combining the significant metabolite list from two different, independent methods) provided a list of metabolites that serve as a strong classifier of healthy vs. malaria samples. In Group 1, for saliva, there were 18 common metabolites, and for urine, there were 24 common metabolites (Table 3). From those, based on the VIP score, the 10 metabolites with the highest score were further selected as potential biomarkers (Table 3, FIGS. 11A-11B). In Group 2, for saliva, there were 13 common metabolites, and for urine, there were 8 common metabolites (Table 4). From those, based on the VIP score, the 10 metabolites with the highest score were further selected as potential biomarkers for saliva and the 8 metabolites for urine were selected (Table 4, FIGS. 11C-11D).

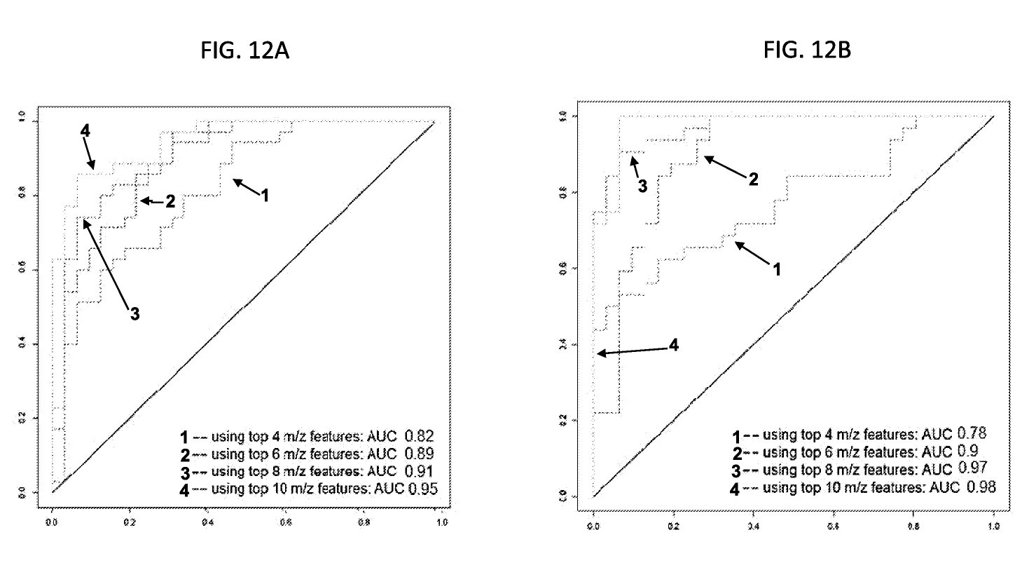

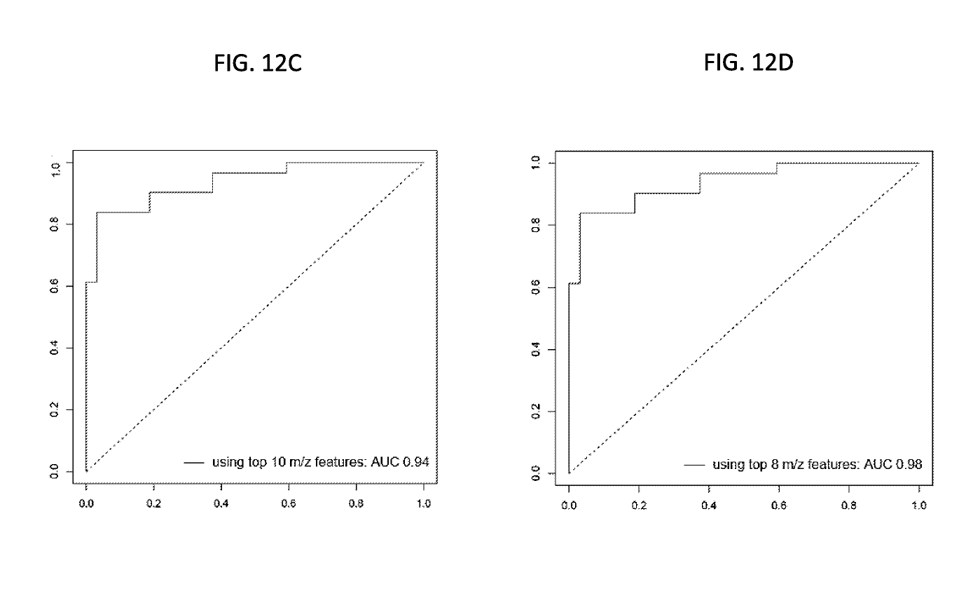

To validate the predictive accuracy of the identified biomarkers, using Receiver Operating Characteristic (ROC) curves (FIGS. 12A-12D), the AUC (Area Under the Curve), the 10-fold AUC accuracy, and the AUC permuted accuracy values were evaluated. The AUC value is calculated by taking into account the sensitivity and the specificity (the true positives, true negatives, false positives and false negatives) of a classifier and it is a way to measure predictive accuracy of this classifier. An AUC value of 1 represents 100% accuracy. In 10-fold cross-validation, the original samples are randomly partitioned into 10 equal size subsets. Of the 10 subsets, a single subset is retained as the validation data for testing the model (the 10 top metabolites in this scenario), and the remaining 9 subsets are used as training data. The cross-validation process is then repeated 10 times (the folds), with each of the 10 subsets used exactly once as the validation data. This process evaluates the generalizability of the model and protects against over-fitting. The permuted AUC accuracy assesses whether the classifier built using the top metabolites (m/z) features has found a real class structure in the data and gives an estimate of robustness of the model/selected features. The null distribution is estimated by permuting (random shuffling) the labels in the data and calculating the permuted AUC accuracy. The process is repeated 100 times and the final permuted AUC accuracy is estimated by taking the average across all iterations. A model built using robust biomarkers ideally has high AUC (and 10-fold AUC accuracy) and low permuted AUC. In other words, the random shuffling of sample class labels should deteriorate the classification accuracy of the model if the selected features are robust biomarkers.

The results indicated the strength and accuracy of the 10 metabolite panel in saliva for Group 1 and Group 2, respectively and the 10 metabolite panel in urine for Group 1 and 8 metabolite panel for Group 2 as a classifier in malaria vs. control samples. The results of the methods and the above values are summarized in FIGS. 12A-12D and Tables 3 and 4.

TABLE 3
Summary of the pipeline of methods used in biomarker discovery (Group
1) in saliva and urine samples from malaria-infected humans.

| Summary of Methods | Saliva | Urine |
|---|---|---|
| a. Metabolite features after 90% presence in | 4,031 | 3,190 |
| control and 90% presence in malaria samples | | |
| b. Metabolite features after regression model: | 133 | 153 |
| associated features in malaria adjusting for | | |
| parasitemia levels, age and gender. Regression | | |
| p-value <0.05 | | |
| c. Metabolite features after PLS classification. | 24 | 31 |
| VIP score >2.5 and fold change >2 | | |
| d. Metabolite features common between b. | 18 | 24 |
| and c. | | |
| e. Metabolite features from d. with the top | 10 | 10 |
| 10 highest VIP score | | |
| AUC (ROC curve) for top 10 features | 0.95 | 0.98 |
| 10-fold AUC accuracy for top 10 features | 0.73 | 0.78 |
| AUC permuted accuracy for top 10 features | 0.64 | 0.67 |

TABLE 4
Summary of the pipeline of methods used in the
novel biomarker discovery (Group 2) in saliva and
urine samples from malaria-infected humans.

| Methods Description | Saliva | Urine |
|---|---|---|
| a. Metabolite features after 35% presence in | 18 | 8 |
| control and 63% presence in malaria samples | | |
| Metabolite features after 63% presence in | 11 | 3 |
| control and 35% presence in malaria samples | | |
| b. Total features from a. | 29 | 11 |
| c. Metabolite features with regression p- | 22 | 8 |
| value <0.05 adjusting for parasitemia, age | | |
| and gender | | |
| d. Metabolite features with PLS VIP score >2.5 | 14 | 9 |
| and fold change >2 | | |
| e. Metabolite features common in c. and d. | 13 | 8 |
| f. Metabolite features with the highest VIP score | 10 | 8 |
| AUC (ROC curve) for top 10 features | 0.94 | 0.98 |
| 10-fold AUC accuracy for top 10 features | 0.76 | 0.81 |
| AUC permuted accuracy for top 10 features | 0.62 | 0.62 |

The identity of the top 10 metabolites from saliva samples from each of Group 1 and Group 2 were predicted (Tables 5 and 6, respectively). The identity of the top 10 metabolites from urine samples from Group 1 and top 8 metabolites from urine samples from Group 2 were predicted (Tables 7 and 8, respectively). All metabolite predictions were estimated based on the given m/z value on positive mode and the following adducts: M+H, M+Na, M+H-2H2O, M+H+H2O, M+ACN+H, and M+2Na-H, the most broad and commonly validated adducts (no any significance measure for the predictions). The METLIN database was used for predictions (available on the World Wide Web at metlin.scripps.edu/index.php). In some cases, there was more than one prediction, as indicated in Tables 5 and 6.

TABLE 5
METLIN database metabolite predictions of top 10 Group 1 metabolites from saliva samples

| | | Putative metabolites | | | | | |
|---|---|---|---|---|---|---|---|
| | time | | | [M + | [M + | [M + | [M + |
| m/z | (sec) | [M + H]⁺ | [M + Na]⁺ | H—2H2O]⁺ | H—H2O]⁺ | ACN + H]⁺ | 2Na—H]⁺ |
| 386.707724 | 65 | — | — | — | — | — | — |
| 604.419416 | 111 | — | — | — | — | Rhodoxan- | |
| | | | | | | thin | |
| | | | | | | Cholesterol | |
| | | | | | | glucuronide | |
| 108.517689 | 244 | — | — | — | — | — | — |
| 286.043791 | 119 | — | Ticlopi- | Flamprop-M | Cyanofenphos | — | Moxonidine |
| | | | dine | Flamprop | | | trans-2-(4- |
| | | | | (±)-Clopidogrel | | | nitrophenyl)- |
| | | | | clopidogrel | | | 3-phenyl- |
| | | | | | | | oxirane |
| | | | | | | | 1,3- |
| | | | | | | | Dihydroxy- |
| | | | | | | | N-methyl- |
| | | | | | | | acridone |
| | | | | | | | N-Benzoyl- |
| | | | | | | | anthranilate |
| | | | | | | | 7-Ethoxy- |
| | | | | | | | resorufin |
| | | | | | | | Mukonidine |
| | | | | | | | Koenigine- |
| | | | | | | | quinone A |
| 227.0114 | 24 | — | CCCP | Dehydro-4- | — | — | — |
| | | | aluminum | methoxy- | | | |
| | | | acetate | cyclobrassinin | | | |
| | | | | 5H-Pyrrolo[3,4- | | | |
| | | | | b]pyrazin-5-one, | | | |
| | | | | 6-(5-chloro-2- | | | |
| | | | | pyridinyl)-6,7- | | | |
| | | | | dihydro-7- | | | |
| | | | | hydroxy- | | | |
| 465.04142 | 87 | — | Piretanide | — | — | O-Des- | — |
| | | | sulfate | | | methyl- | |
| | | | | | | tolrestat | |
| | | | | | | sulfate | |
| 874.64237 | 221 | — | — | — | C24 | — | — |
| | | | | | Sulfatide | | |
| 529.865684 | 104 | — | — | — | — | — | — |
| 758.009261 | 133 | — | — | — | — | — | — |
| 85.0285911 | 91 | 3-; 4- | — | Succinic acid | — | — | — |
| | | Hydroxy- | | semialdehyde | | | |
| | | 2-butynal | | Acetoacetic acid | | | |
| | | 2(5H)- | | 3-methyl | | | |
| | | Furanone | | pyruvic acid | | | |
| | | 2(3H)- | | 2-Methyl-3- | | | |
| | | Furanone | | oxopropanoic | | | |
| | | | | acid | | | |
| | | | | 4-hydroxy- | | | |
| | | | | crotonic acid | | | |
| | | | | (S)-Methyl- | | | |
| | | | | malonic acid | | | |
| | | | | semialdehyde | | | |
| | | | | 2-methyl-3-oxo- | | | |
| | | | | propanoic acid | | | |
| | | | | Acetic anhydride | | | |

TABLE 6
METLIN database metabolite predictions of top 10 Group 2 metabolites from
saliva samples

| | | Putative metabolites | | | | | |
|---|---|---|---|---|---|---|---|
| | time | [M + | [M + | [M + | [M + | [M + | [M + |
| m/z | (sec) | H]⁺ | Na]⁺ | H—2H2O]⁺ | H—H2O]⁺ | ACN + H]⁺ | 2Na—H]⁺ |
| 1122.678802 | 107 | — | — | — | — | — | — |
| 583.866644 | 101 | — | — | — | — | — | — |
| 112.0107116 | 143 | — | — | — | — | — | — |
| 427.7743432 | 119 | — | — | — | — | — | — |
| 723.3652248 | 117 | — | — | Marshdimerin | — | — | — |
| 1030.147415 | 66 | — | — | — | — | — | — |
| 952.6397276 | 98 | — | — | — | — | — | — |
| 924.3574519 | 67 | — | — | — | — | — | — |
| 524.4171783 | 23 | — | — | — | — | — | — |
| 963.1861344 | 67 | — | — | — | — | 2-Naphth- | — |
| | | | | | | oyl-CoA | |

TABLE 7
METLIN database metabolite predictions of top 10 Group 1 metabolites from urine samples

| | Putative metabolites | | | | | | |
|---|---|---|---|---|---|---|---|
| | time | | | [M + | [M + | [M + | [+M + |
| m/z | (sec) | [M + H]⁺ | [M + Na]⁺ | H—2H2O]⁺ | H—2H2O]⁺ | ACN + H]⁺ | 2Na-H]⁺ |
| 203.99138 | 258 | — | 2-Methyl | — | — | — | — |
| | | | thiobenzo | | | | |
| | | | thiazole | | | | |
| 305.16444 | 66 | — | 10-Hydroxy | YOHIMBIC | 4-hydroxy | (E,E)- | — |
| | | | desipramin | ACID | Nonenal | Lansamide I | |
| | | | 2-Hydroxy | 3-Hydroxy | Mercapturic | Girinimbine | |
| | | | desmethyl | quinine | Acid-d3 | Lansium | |
| | | | imipramine | 11-Hydroxy | PtdIns-(1- | amide B | |
| | | | AG-17 | tubotaiwine | arachidonoyl-d8, | | |
| | | | | 2′- | 2-arachidonoyl) | | |
| | | | | Oxoquinidine | Akuammicine | | |
| | | | | Quinine-N′- | Gelsemine | | |
| | | | | Oxide | Gardneral | | |
| | | | | 3-Hydroxy | Quinidinone | | |
| | | | | quinidine | 4-Chloro | | |
| | | | | Quinidine | testosterone | | |
| | | | | N′-oxide | 16beta- | | |
| | | | | Quinine | Chloro- | | |
| | | | | 10,11- | 17beta- | | |
| | | | | epoxide | hydroxyandrost- | | |
| | | | | Quinine-N- | 4-en-3-one | | |
| | | | | Oxide | 19-Chloro- | | |
| | | | | | 17beta- | | |
| | | | | | hydroxyandrost- | | |
| | | | | | 4-en-3-one | | |
| 214.08772 | 63 | Benzyl | Trihomomethionine | — | — | Menadione | — |
| | | nicotinate | | | | 1-Naphthoic acid | |
| | | Fenamic acid | | | | Vitamin K3 | |
| | | Salicylanilide | | | | Dehydromatricaria | |
| | | 2-(4-Methyl- | | | | ester | |
| | | 5-thiazol- | | | | (Z)-2-Decene- | |
| | | yl)ethyl | | | | 4,6,8-triynoic acid | |
| | | butanoate | | | | methylester | |
| | | 2-(4-Methyl- | | | | 1-Hydroxy- | |
| | | 5-thiazoly) | | | | 2-naphthaldehyde | |
| | | ethyl | | | | 2-Naphthoic acid | |
| | | isobutyrate | | | | 3Z-Undecene- | |
| | | | | | | 5,7,10-triynoic | |
| | | | | | | acid | |
| | | | | | | 4E-Undecene- | |
| | | | | | | 6,8,10-triynoic | |
| | | | | | | acid | |
| 179.04712 | 277 | L-Cys-Gly | 1-Naphthaldehyde | (S)-ACPA | — | — | (+/−)-3- |
| | | Cysteinyl- | 2-Naphthaldehyde | | | | (Ethylthio) |
| | | Glycine | | | | | butanol |
| | | Glycyl- | | | | | 2-Mercapto- |
| | | Cysteine | | | | | 2-methyl-1- |
| | | | | | | | pentanol |
| | | | | | | | (+/−)-4- |
| | | | | | | | Mercapto- |
| | | | | | | | 4-methyl- |
| | | | | | | | 2-pentanol |
| | | | | | | | 3-Mercapto- |
| | | | | | | | 2-methyl |
| | | | | | | | pentanol |
| | | | | | | | 4-Methoxy- |
| | | | | | | | 2-methyl- |
| | | | | | | | 2-butane |
| | | | | | | | thiol |
| | | | | | | | 3-Mercapto- |
| | | | | | | | 1-hexanol |
| 387.92813 | 138 | — | — | — | — | — | — |
| 117.95964 | 268 | — | — | — | — | — | — |
| 338.08648 | 73 | 2,8- | Flusilazole | DIMBOA- | 2,5-Diamino- | N-Acetyl | Glutamyl- |
| | | Dihydroxy | | glucoside | 6-(5- | djenkolic acid | Phenylalanine |
| | | quinoline- | | DIMBOA- | phospho-D- | Disulfiram | Phenylalanyl- |
| | | beta-D- | | Glc | ribitylamino) | Phaseolic acid | Glutamate |
| | | glucuronide | | | pyrimidin- | Mono-trans-p- | N,N′- |
| | | | | | 4(3H)-one | coumaroyl- | (acridine-3,6- |
| | | | | | | mesotartaric acid | diyl)diacetamide |
| | | | | | | 2-O- | GYKI 52466 |
| | | | | | | Feruloyltartronic | 7-Aceta- |
| | | | | | | acid | midonitrazepam |
| | | | | | | cis-Coutaric acid | Desethyl- |
| | | | | | | | enenorfloxacin |
| 682.79589 | 117 | — | — | — | — | — | — |
| 113.05889 | 52 | Sorbic acid | | | | | |
| | | Aleprolic acid | | | | | |
| | | 3,5-hexadienoic | | | | | |
| | | acid | | | | | |
| | | 5-hexynoic acid | | | | | |
| | | trans-1,2- | | | | | |
| | | Dihydrobenzene- | | | | | |
| | | 1,2-diol | | | | | |
| | | 4-oxo-2E- | | | | | |
| | | Hexenal | | | | | |
| | | 4-oxo-2Z- | | | | | |
| | | Hexenal | | | | | |
| | | Cyclohexane- | | | | | |
| | | 1,3-dione | | | | | |
| | | Cyclohexane- | | | | | |
| | | 1,2-dione | | | | | |
| | | 1,4-Cyclohex- | | | | | |
| | | anedione | | | | | |
| | | Parasorbic acid | | | | | |
| | | C6:2n-1,3 | | | | | |
| | | 3-Hexynoic acid | | | | | |
| | | 4-Hexynoic acid | | | | | |
| | | 5-Hexynoic acid | | | | | |
| | | 5-Hexyn-1-oic | | | | | |
| | | acid | | | | | |
| | | 2-hexenedial | | | | | |
| | | 3-hexenedial | | | | | |
| | | 6-hydroxy- | | | | | |
| | | 2,4-hexadienal | | | | | |
| | | 5,5-Dimethyl- | | | | | |
| | | 2(5H)-furanone | | | | | |
| | | 2,5-Dimethyl- | | | | | |
| | | 3(2H)-furanone | | | | | |
| | | Syoyualdehyde | | | | | |
| | | 2-(Methoxy | | | | | |
| | | methyl)furan | | | | | |
| | | xi-3,5-Dimethyl- | | | | | |
| | | 2(5H)-furanone | | | | | |
| | | 5,5-Dimethyl- | | | | | |
| | | 2(5H)-furanone | | | | | |
| | | 2-hydroxy-3- | | | | | |
| | | methyl-2- | | | | | |
| | | Cyclopenten- | | | | | |
| | | 1-one | | | | | |
| 726.72644 | 111 | — | — | — | — | — | — |

TABLE 8
METLIN database metabolite predictions of top 8 Group 2 metabolites from urine samples

| | Putative metabolites | | | | | | |
|---|---|---|---|---|---|---|---|
| | time | | | [M + | [M + | [M + | [+M + |
| m/z | (sec) | [M + H]⁺ | [M + Na]⁺ | H—2H2O]⁺ | H—2H2O]⁺ | ACN + H]⁺ | 2Na-H]⁺ |
| 692.3898116 | 26 | Fumonisin | — | — | — | — | — |
| | | C2 | | | | | |
| | | Fumonisin | | | | | |
| | | C3 | | | | | |
| 271.9162641 | 179 | — | — | — | — | — | — |
| 288.1305573 | 85 | — | — | Naftifine | — | Asparaginyl- | Lysyl- |
| | | | | hydrochloride | | Asparagine | Proline |
| | | | | | | N2-Oxalyl | Prolyl- |
| | | | | | | arginine | Lysine |
| 954.3185803 | 67 | — | — | — | — | — | — |
| 352.0259716 | 66 | Oxine- | Iprodione | — | — | — | dCMP |
| | | copper | | | | | |
| 260.0110747 | 165 | — | — | Riluzolamide | — | — | — |
| 646.7783966 | 73 | — | — | — | — | — | — |
| 674.4584899 | 73 | — | — | PE(14:0/20:5 | — | — | — |
| | | | | (5Z, 8Z, 11Z, | | | |
| | | | | 14Z, 17Z)) | | | |
| | | | | PE(14:1(9Z)/ | | | |
| | | | | 20:4(5Z, 8Z, | | | |
| | | | | 11Z, 14Z)) | | | |
| | | | | PE(14:1(9Z)/ | | | |
| | | | | 20:4(8Z, 11Z, | | | |
| | | | | 14Z, 17Z)) | | | |
| | | | | PE(16:1(9Z)/ | | | |
| | | | | 18:4(6Z, 9Z, | | | |
| | | | | 12Z, 15Z)) | | | |
| | | | | PE(18:4(6Z, | | | |
| | | | | 9Z, 12Z, 15Z)/ | | | |
| | | | | 16:1(9Z)) | | | |
| | | | | PE(20:4(5Z, | | | |
| | | | | 8Z, 11Z, 14Z)/ | | | |
| | | | | 14:1(9Z)) | | | |
| | | | | PE(20:4(8Z, | | | |
| | | | | 11Z, 14Z, 17Z)/ | | | |
| | | | | 14:1(9Z)) | | | |
| | | | | PE20:5(5Z, | | | |
| | | | | 8Z, 11Z, 14Z, | | | |
| | | | | 17Z)/14:0) | | | |

### Example 4

### Comparison of Metabolites Identified in Culture, Saliva, and Urine Samples

The fold-change of the metabolites identified in Tables 1 and 2 were determined in saliva and urine samples from malaria-infected samples compared to control samples (Tables 9 and 10). In saliva samples, the m/z 140.07 feature was significantly increased and sphingosine was significantly decreased compared to non-infected controls. In urine samples, thiamine was significantly decreased and the m/z 209/07 feature was significantly increased compared to non-infected controls.

TABLE 9
Metabolites in saliva and urine in comparison to the metabolites originally identified in culture supernatant during Plasmodium infection

| | SALIVA | SALIVA | SALIVA | URINE | URINE | URINE |
|---|---|---|---|---|---|---|
| Metabolite | (m/z) | fold change | p-value (t-test) | (m/z) | fold change | p-value (t-test) |
| 3-methyl | — | Not detected | — | 132.08105 | 3.29700 | 0.06400 |
| indole | | | | | | |
| (m/z 132.0811 | | | | | | |
| [M + H]) | | | | | | |
| Succinyl | — | Not detected | — | 181.04753 | 0.10369 | 0.48205 |
| acetone | | | | | | |
| (m/z 181.0481 | | | | | | |
| [M + Na]) | | | | | | |
| O-arachidonoyl | — | Not detected | — | 361.27154 | −2.92308 | 0.13123 |
| glycidol | | | | | | |
| (m/z 361.2715 | | | | | | |
| [M + H]) | | | | | | |
| Thiamine | 265.11197 | Detected but not at | — | 265.11866 | −0.44416 | 0.01765 |
| (m/z 265.1118 | | 50% presence | | | | |
| [M + H]) | | | | | | |
| Arginine | 175.11938 | −0.37450 | 0.28722 | 175.11931 | 0.06258 | 0.38193 |
| (m/z 175.119 | | | | | | |
| [M + H]) | | | | | | |
| Linoleic acid | 281.24801 | −0.08622 | 0.96746 | — | Not detected | — |
| (m/z 281.2475 | | | | | | |
| [M + H]) | | | | | | |
| m/z 133.10 | 133.1014786; | −0.664930243875002; | 0.322621534075598; | 133.1013877; | −0.121565946048161; | 0.226843678015273; |
| | 133.1055486 | −0.11849604557143 | 0.698905180235666 | 133.1054383 | −0.01007892 | 0.478717607 |
| m/z 140.07 | 140.07061 | 3.39205 | 0.02068 | 140.07079 | −0.17361 | 0.46062 |
| m/z 144.98 | 144.9824974; | 1.01921608308929; | 0.239262048020948; | 144.982311; | −0.200954974955419; | 0.0609514037526147; |
| | 144.9825006 | 1.17695042353571 | 0.442668126405955 | 144.9823998 | −0.12290201566206 | 0.17280332475016 |
| m/z 146.98 | 146.98072 | 0.85566 | 0.34624 | 146.98058 | −0.01059 | 0.46899 |
| m/z 152.04 | — | Not detected | — | 152.04317 | 0.91926 | 0.35434 |
| m/z 173.03 | 173.03058 | −0.10054 | 0.96419 | 173.0303716; | −3.17461112164026; | 0.119285669965963; |
| | | | | 173.0397564; | −3.35219380949589; | 0.0671078814493341; |
| | | | | 173.0300778 | −0.216889932144554 | 0.452333582553186 |
| m/z 181.01 | 181.01004 | −1.93470 | 0.45824 | 181.0146935; | −0.0633819517539429; | 0.291257077113314; |
| | | | | 181.0152584 | −0.199399599768736 | 0.135357465194568 |
| m/z 183.01 | 183.01031 | −0.72812 | 0.55432 | 183.01018 | 0.81909 | 0.30925 |
| m/z 191.04 | 191.04044 | Detected but not at | — | — | Not detected | — |
| | | 50% presence | | | | |
| m/z 205.02 | — | Not detected | — | — | Not detected | — |
| m/z 205.96 | 205.96198 | Detected but not at | — | 205.96220 | Detected but not at | — |
| | | 50% presence | | | 50% presence | |
| m/z 209.07 | 209.07308 | 0.11200 | 0.67685 | 209.0730741; | 3.97179761535571; | 0.0426342794783669; |
| | | | | 209.0787901 | 0.313193553133324 | 0.408468513331127 |
| m/z 222.10 | 222.1258595; | 1.12532269544643; | 0.667767291945339; | 222.11268 | −0.18401 | 0.06273 |
| | 222.1126373 | −4.19176220461608 | 0.0527324153275404 | | | |
| m/z 222.888 | — | Not detected | — | 222.88896 | Detected but not at | — |
| | | | | | 50% presence | |
| m/z 231.97 | — | Not detected | — | 231.97257 | −0.93858 | 0.35697 |
| m/z 232.09 | 232.09514 | 0.06909 | 0.68761 | 232.09694 | Detected but not at | — |
| | | | | | 50% presence | |
| m/z 263.92 | 263.92675 | −2.60194 | 0.20103 | — | Not detected | — |
| m/z 504.83 | 504.83541 | 3.01878 | 0.14172 | — | Not detected | — |
| m/z 546.40 | 546.41662 | 0.97395 | 0.66890 | 546.45284 | Detected but not at | — |
| | | | | | 50% presence | |

The metabolite was up-regulated in malaria samples if the fold change is >0 and down-regulated if the fold change is <0. Where multiple metabolites were identified within the specified m/z range, they are separated by a “;”. Features that were not detected in saliva or urine samples or were not analyzed are not listed.

TABLE 10
Metabolites in saliva and urine in comparison to the metabolites originally identified in cell pellet during
Plasmodium infection

| | | | SALIVA | | | URINE |
|---|---|---|---|---|---|---|
| | SALIVA | SALIVA | p-value | URINE | URINE | p-value |
| Metabolite | (m/z) | fold change | (t-test) | (m/z) | fold change | (t-test) |
| Arginine | 175.1193817 | −0.374502245 | 0.287215502 | 175.1193111 | 0.062578476 | 0.381929971 |
| (m/z 175.119 | | | | | | |
| [M + H]) | | | | | | |
| Phosphatidylcholine | 132.1022336 | 0.153262432 | 0.594664055 | 132.1021135; | 0.05611; | 0.36567; |
| (m/z 132.10188 | | | | 132.1214094 | −0.0973 | 0.33018 |
| [M + H]) | | | | | | |
| Sphingosine | 300.2898247 | −4.029083495 | 0.051440698 | 300.2899491 | Detected but | — |
| (m/z 300.2897 | | | | | not at 50% | |
| [M + H]) | | | | | presence | |
| m/z 248.16 | 248.1609747 | Detected but not at | — | 248.1681402 | −0.147320612 | 0.373574895 |
| | | 50% presence | | | | |
| m/z 258.89 | 258.8992081 | −0.136505983 | 0.527807524 | 258.8988604 | −0.707451679 | 0.218070167 |
| m/z 301.29 | 301.2931553 | Detected but not at | — | — | Not detected | — |
| | | 50% presence | | | | |
| m/z 468.30 | 468.3042549; | −1.85680618251785; | 0.129582506 | 468.3895552 | 1.873663829 | 0.22487997 |
| | 468.3027137 | −1.85731836405357 | | | | |
| m/z 495.33 | 495.3334813 | 0.274181046 | 0.380066775 | — | Not detected | — |

The metabolite was up-regulated in malaria samples if the fold change is >0 and down-regulated if the fold change is <0. Where multiple metabolites were identified within the specified m/z range, they are separated by a “;”. Features that were not detected in saliva or urine samples or were not analyzed are not listed.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1. A method of detecting presence of Plasmodium in a subject, comprising:
obtaining a sample from the subject;
analyzing the sample from the subject by mass spectrometry, liquid chromatography, immunoassay, aptamer assay methods, or a combination of two or more thereof, to detect an amount of one or more metabolites in any one of Table 7, Table 5, Table 8, Table 6, Table 1, or Table 2;
comparing the amount of the one or more metabolites to a reference value or to the amount of the one or more metabolites in a control sample obtained from a non-Plasmodium-infected subject, an in vitro non-Plasmodium-inoculated culture, a pooled sample from non-Plasmodium-infected subjects, and/or a reference value from a non-Plasmodium-infected subject or subjects, wherein an increase of at least about 1.2 fold or a decrease of at least about 10% in the amount of the one or more metabolites compared to the referenced value or the control sample indicates presence of a Plasmodium infection in the subject from which the sample was obtained; and
determining presence of Plasmodium in the subject if the amount of the one or more metabolites is different than the control.

2. The method of claim 1, wherein detecting the amount of one or more metabolites comprises detecting the amount of five or more metabolites in Table 7, five or more metabolites in Table 5, five or more metabolites in Table 8, five or more metabolites in Table 6, or a combination thereof.

3. The method of claim 2, wherein detecting the amount of one or more metabolites comprises detecting the amount of each metabolite in Table 7, each metabolite in Table 5, each metabolite in Table 8, and/or each metabolite in Table 6.

4. The method of claim 1, wherein the step of analyzing the sample from the subject comprises:
detecting an amount of one or more metabolites in Table 1 and/or Table 2 in the sample from the subject, and wherein one or more of the metabolites is not 3-methylindole, succinylacetone, S-methyl-L-thiocitrulline, O-arachidonoyl glycidol, isoleucine, or arginine.

5. The method of claim 4, wherein detecting the amount of one or more metabolites comprises detecting the amount of five or more metabolites in Table 1, Table 2, or a combination thereof.

6. The method of claim 5, wherein detecting the amount of one or more metabolites comprises detecting the amount of each of the metabolites in Table 1 and/or each of the metabolites in Table 2.

7. The method of claim 1, wherein detecting presence of Plasmodium in the subject indicates that the subject is infected with Plasmodium.

8. The method of of claim 1, wherein the step of analyzing the sample from the subject comprises:
detecting an amount of five or more metabolites in any one of Table 7, Table 5, Table 8, Table 6, Table 1, or Table 2—in the sample from the subject.

9. The method of claim 8, wherein detecting the amount of five or more metabolites comprises detecting the amount of each of the metabolites in Table 7, each of the metabolites in Table 5, each of the metabolites in Table 8, each of the metabolites in Table 6, each of the metabolites in Table 1, and/or each of the metabolites in Table 2.

10. The method of claim 1, wherein the sample from the subject is one or more of blood, plasma, serum, urine, saliva, sweat, cerebrospinal fluid, middle ear fluid, breast milk, bronchoalveolar lavage, tracheal aspirate, sputum, tears, mucous, oral fluid, nasopharyngeal aspirate, oropharyngeal aspirate, oral swab, eye swab, cervical swab, vaginal swab, rectal swab, stool or stool suspension.

11. (canceled)

12. The method of claim 1, wherein analyzing the sample from the subject is by linear quadrupole ion trap Fourier transform mass spectrometry coupled with C18 liquid chromatography or by high-field hybrid quadrupole mass spectrometry coupled with C18 liquid chromatography.

13. (canceled)

14. The method of claim 1, further comprising administering to the subject one or more anti-malarial therapeutic agents if the amount of the one or more metabolites is different than the control.

15. The method of claim 14, wherein the anti-malarial therapeutic agent comprises artemisinin a derivative thereof, an artemisin-based combination therapy, atovaquone-proguanil, chloroquine, primaquine, mefloquine, quinine, or a combination of two or more thereof.

16. The method of claim 15, wherein the artemisinin or a derivative thereof is artesunate, dihydroartemisinin, or artemether.

17. The method of claim 1, wherein analyzing the sample from the subject is by an aptamer assay that comprises:
contacting the sample from the subject with an aptamer under conditions sufficient to form a binding complex between the aptamer and one or more metabolites in any one of Table 7, Table 5, Table 8, Table 6, Table 1, or Table 2; and
detecting presence of one or more binding complexes between the aptamer and the one or more metabolites by one or more of Western blotting, ELISA, radioimmunoassay, fluorescence microscopy or flow cytometry.

18. The method of claim 17, further comprising using a detectable label, wherein the detectable label is one or more of a radiolabel, fluorophore or enzyme.

19. The method of claim 18, further comprising determining quantity of the one or more binding complexes between the aptamer and the one or more metabolites.

20. The method of claim 10, further comprising processing the sample by adding one or more of a solvent, preservative, additive, buffer or a combination thereof to the sample from the subject, or reducing or removing cells from the sample by centrifugation or gravity.

21. The method of claim 1, wherein the subject is a human or a non-human mammal.

22. The method of claim 1, wherein the increase in the amount of the one or more metabolites is at least about 1.5-fold, at least about 2-fold, at least about 3-fold, at least about 5-fold, at least about 10-fold, at least about 20-fold, at least about 50-fold, at least about 100-fold, at least about 500-fold, or at least about 1000-fold compared to the control.

23. The method of claim 1, wherein the decrease in the amount of the one or more metabolites is at least about 15%, at least about 20%, at least about 25%, at least about 30%, at least about 40%, at least about 50%, at least about 60%, at least about 70%, at least about 80%, at least about 90%, or at least about 95% compared to the control.

24. The method of claim 1, wherein the one or more metabolites comprise 2-methylbenzothiazole.
